(19) United States
(12) Patent Application Publication
(10) Pub. No.: US 2023/0126576 A1
(43) Pub. Date: Apr. 27, 2023

(54) SYSTEMS AND METHODS FOR ACCELERATION-BASED SEIZURE DETECTION

(71) Applicant: LivaNova USA, Inc., Houston, TX (US)

(72) Inventor: Gerrard Merrill Carlson, Houston, TX (US)

(21) Appl. No.: 17/508,539

(22) Filed: Oct. 22, 2021

(51) Int. Cl. A61N 1/36

(52) U.S. Cl. CPC A61N 1/36064; A61N 1/36031; A61N 1/36135; A61B 2562/0219

(57) ABSTRACT

A seizure detection system comprises a sensor and a control system. The sensor is configured to detect acceleration data associated with a patient’s movements. The control system is programmed to monitor, via the sensor, the acceleration data to detect a seizure precursor event. The control system is further programmed to, upon detection of the seizure precursor event, perform a frequency analysis of the acceleration data to identify a frequency of the patient’s movements. The control system is further programmed to determine that the patient is experiencing a seizure event based on the frequency of the patient’s movements.

### BACKGROUND

The effective management of epilepsy necessitates reliable long-term (over days and months) monitoring of seizures. Although visual inspection of electroencephalographic (EEG) signals is the current gold standard for seizure detection in supervised environments, such as the epilepsy monitoring unit or the intensive care unit, it is not practical to use this approach to objectively quantify long-term seizure frequency. The current approach to track long-term seizure frequency is by maintaining seizure diaries. However, it has been shown that self-reporting of seizure incidence is severely inaccurate. Tonic-clonic seizures are thought to be the leading risk factor for sudden unexpected death in epilepsy (SUDEP). There is a need for the automatic detection of tonic-clonic seizure to decrease morbidity and mortality associated with seizures; especially at night-time hours.

### SUMMARY

One embodiment relates to a seizure detection system comprising a sensor and a control system. The sensor is configured to detect acceleration data associated with a patient’s movements. The control system is programmed to monitor, via the sensor, the acceleration data to detect a seizure precursor event. The control system is further programmed to, upon detection of the seizure precursor event, perform a frequency analysis of the acceleration data to identify a frequency of the patient’s movements. The control system is further programmed to determine that the patient is experiencing a seizure event based on the frequency of the patient’s movements.

Another embodiment relates to a method of detecting a seizure event of a patient. The method comprises monitoring, by a sensor, acceleration data associated with the patient’s movements to detect a seizure precursor event. The method further comprises, upon detection of the seizure precursor event, performing a frequency analysis of the acceleration data to identify a frequency of the patient’s movements. The method further comprises determining whether the patient is experiencing the seizure event based on the frequency of the patient’s movements.

Another embodiment relates to one or more non-transitory computer-readable mediums including instructions executable by one or more processors. The instructions are executable by the one or more processors to monitor, via a sensor, acceleration data associated with a patient’s movements to detect a seizure precursor event. The instructions are further executable by the one or more processors to, upon detection of the seizure precursor event, perform a frequency analysis of the acceleration data to identify a frequency of the patient’s movements. The instructions are further executable by the one or more processors to determine that the patient is experiencing a seizure event based on the frequency of the patient’s movements.

### BRIEF DESCRIPTION OF THE DRAWINGS

Further features, characteristics, and advantages of the present disclosure will become apparent to a person of ordinary skill in the art from the following detailed description of embodiments of the present disclosure, made with reference to the drawings annexed, in which like reference characters refer to like elements.

FIG. 1 is a schematic view of a seizure detection system utilizing an implanted medical device, according to an exemplary embodiment.

FIG. 2 is a schematic view of the seizure detection system of FIG. 1, utilizing a wearable medical device, according to an exemplary embodiment.

FIG. 3 is a schematic view of a medical device configured for use with the seizure detection system of FIG. 1, according to an exemplary embodiment.

FIG. 4 is a flowchart of a method for detecting seizure events using a multi-stage movement event of interest (MEOI) detection algorithm, according to an exemplary embodiment.

FIG. 5 is a plot of the patient’s posture, activity level, and movement frequency throughout the method for detecting seizure events of FIG. 4, according to an exemplary embodiment.

FIG. 6 is a plot of the patient’s activity level throughout the method of FIG. 4, showing various timers associated with the multi-stage MEOI detection algorithm, according to an exemplary embodiment.

FIG. 7A is a first portion of a flow diagram for a process of obtaining and analyzing acceleration data obtained from an acceleration sensor to detect seizure events, according to an exemplary embodiment.

FIG. 7B is a second portion of the flow diagram of FIG. 7A, according to an exemplary embodiment.

FIG. 7C is a third portion of the flow diagram of FIG. 7A, according to an exemplary embodiment.

FIG. 8 is a flow diagram of a factory calibration procedure for an acceleration sensor, according to an exemplary embodiment.

FIG. 9 is a flow diagram of an upright and supine calibration procedure for an acceleration sensor, according to an exemplary embodiment.

FIG. 10 is a flow diagram of a posture detection procedure for a medical device, according to an exemplary embodiment.

FIGS. 11-16 are various flow graphs of special N-point radix 2 real decimation in time (DIT) discrete Fourier transform (DFT) analyses, according to exemplary embodiments.

FIG. 17 is a frequency plot showing an output magnitude of a frequency analysis for various frequencies, according to an exemplary embodiment.

FIG. 18 is a plot showing a number of true positive seizure detections across a range of minimum seizure detection periods for various activity time period thresholds, according to an exemplary embodiment.

FIG. 19 is a plot showing a number of false positive seizure detections across a range of minimum seizure detection periods for various activity time period thresholds, according to an exemplary embodiment.

### DETAILED DESCRIPTION

Seizure detection via detection of autonomic signatures such as motor signals that are altered by seizures presents itself as a viable alternative for long-term monitoring of seizures. This approach becomes even more attractive for monitoring the pediatric epilepsy population, especially during the night when supervision is reduced and the risk of SUDEP is high. Wearable devices to chronically monitor motor signals associated with seizures may be utilized with greater ease than EEG-based devices and can significantly improve the overall quality of life of patients and caregivers, as well as provide an objective way for physicians to track their patients’ seizures. With use of accelerometry, seizures that express themselves in movements or seizures that disturb normal movement patterns can be detected. An advantage of accelerometers is that they can be worn on the body, resulting in more freedom to pursue normal daily activities. Another advantage of accelerometers is that it is possible to observe movements under blankets unlike other approaches such as video (e.g., used in baby monitors).

Seizures in which the main clinical manifestations are movements are called motor seizures. These motor seizures can be divided into two major subgroups, simple motor seizures and complex motor seizures. In simple motor seizures, motor movements are relatively ‘simple’ and unnatural and are caused by a relatively massive discharge in the motor structures of the cortex. Complex motor seizures are seizures in which the movements are relatively complex and simulate natural movement, except that they are inappropriate for the situation. These seizures often arise from the limbic system.

The movements during simple motor seizures tend to be stereotypical and, when movements are repetitive, they affect the same body segment. Simple motor seizures can be subdivided into the following types: myoclonic, clonic, tonic, and tonic-clonic seizures. These seizure types depend on the duration of the muscle contractions, the frequency of movement repetition, and the muscles involved. A myoclonic seizure is characterized by a sudden jerk. Each myoclonic jerk typically involves only a few adjacent muscles (e.g., only one antagonistic pair). Myoclonic jerks are usually generated by activation of the primary or secondary motor areas in the cortex by epileptic discharges. Most of these epileptic seizures begin in the frontal lobe and then spread. The duration of this train of muscle action potentials is less than 100 ms and the frequency is 50 Hz. Further, only one limb is typically involved in myoclonic seizures. An accelerometer can thus detect myoclonic seizures by detecting an instantaneous changes in the tone.

Clonic seizures generally include repeated myoclonic contractions that regularly recur at intervals between 0.2 and 5 times per second. During a clonic seizure, the affected parts of the body show repetitive jerking. Accelerometers placed on the chest and limbs are able to sense this repetitive jerking motion.

During tonic seizures a sudden sustained contraction of multiple muscle groups is typically seen. Often the limbs undergo a slow change of posture. However, depending on how quickly the seizure starts, tonic seizures can also begin with a massive jerk. Tonic seizures have a duration that lies typically between 10 and 20 seconds, but can vary between 2 seconds and 60 seconds. Accelerometers on the limbs and chest in these instances may detect higher frequencies between 2 Hz to 5 Hz.

Tonic-clonic seizures consist of an initial tonic phase during which the patient has the legs and arms in extension with the arms adducted and crossed in front of the body. The tonic phase typically lasts between 5 and 10 seconds and is then followed by a series of tremor-like muscle contractions. The movements of the arms increase progressively in amplitude as the repetition rate diminishes. Eventually, this generally evolves into clonic contraction (flexions at the elbow), initially having a frequency of 5 Hz, and then progressively decreasing in frequency to one contraction every 1 to 3 seconds.

At the same time, normal active physiological movements, such as walking and running, may similarly manifest with periodic frequencies between 0.5 Hz and 6 Hz. However, in some instances, the level of physical exertion can be significantly higher than those during seizures. Moreover, these movements are well-coordinated as opposed to the ones produced during seizures.

Using this information, the systems and methods described herein utilize an algorithm developed for automatic classification of movements that are physiological versus those that may be pathological. Based on a review of features of day-to-day physiological movements and pathological movements associated with seizures, most of the movements can be clearly described using measures of intensity and frequency of movement. Thus, the present disclosure provides systems and methods utilizing an algorithm that distinguishes physiological movements from pathological movements by a) extracting patient posture and movement features using one or more acceleration sensors and b) distinguishing physiological movements from pathological movements based on these features.

Specifically, the systems and methods described herein make it possible to detect the occurrence of an abnormal night time patient movement that may associated with an epileptic seizure (i.e., a movement event of interest indicative of a seizure event). As such, the systems and methods described herein may be particularly applicable for use with patients at risk of SUDEP. That is, patient seizures (e.g., generalized tonic-clonic and clonic seizures) may be particularly lethal. Studies directed toward pediatric and adult SUDEP patient populations show that epileptic patients often die in their sleep after having an unwitnessed tonic-clonic, clonic or generalized tonic-clonic seizure event. The systems and methods described herein allow for a care provider, typically the patient’s parent, to be alerted when the patient is experiencing a seizure event to allow for appropriate care to be administered to the patient. Further, in some instances, the systems and methods described herein may further allow for automated vagus nerve stimulation (VNS) to be applied to the patient upon detection of a seizure event.

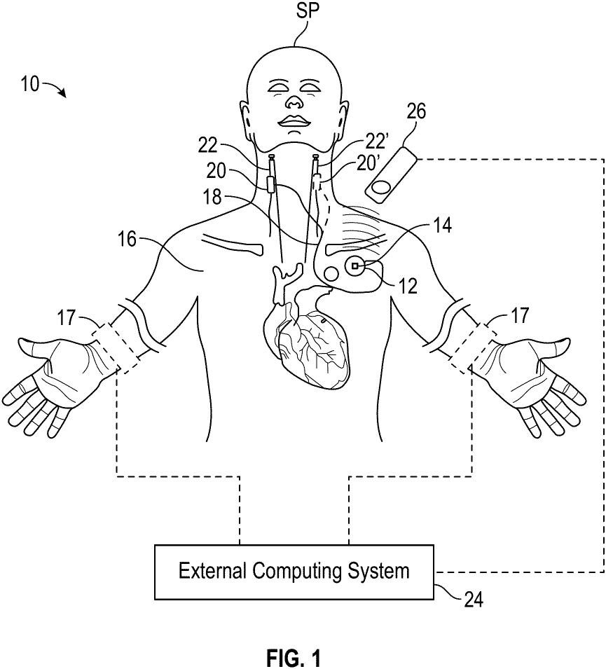

Shown in FIG. 1 is a seizure detection system 10 including a medical device 12 for implementing one or more embodiments of the present disclosure. For example, in some embodiments, the medical device 12 is configured to detect seizure events in a subject patient SP and, in particular, to monitor the patient’s posture and movement to detect and analyze motion events of interest to determine whether the patient is experiencing a seizure event. In some embodiments, the medical device 12 may be further configured to detect the patient having a seizure and to apply therapeutic vagus nerve stimulation to the patient in response to the detected seizure. In some embodiments, the medical device 12 is configured to additionally or alternatively provide seizure alerts to caregivers and/or physicians and/or to store a log of detected seizures and associated physiological information to aid in patient assessment over time.

Specifically, the medical device 12 is configured to capture patient posture and movement data using one or more acceleration sensors 14 and utilize a multi-stage movement events of interest (MEOI) detection algorithm to effectively detect seizure events, while also minimizing processing and power consumption requirements.

For example, in some embodiments, the multi-stage MEOI detection algorithm is configured to continuously monitor a posture of the patient. Upon determining that the patient has been in a recumbent position for a predetermined period of time, the multi-stage MEOI detection algorithm is then configured to trigger a monitoring of the patient’s activity level compared to a first patient activity threshold. Upon determining that the patient’s activity level has been below the patient activity threshold for a predetermined amount of time, the multi-stage MEOI detection algorithm is then configured to continue monitoring the patient’s activity level compared to a second patient activity threshold. Upon determining that the patient’s activity level has been above the patient activity threshold for a predetermined amount of time, the multi-stage MEOI detection algorithm is then configured to trigger a frequency analysis of the patient’s movements to determine whether the patient is experiencing a seizure event.

By only performing the computationally-intensive frequency analysis of the patient’s movements after the patient has been (1) recumbent for a predetermined period of time, then (2) inactive for a predetermined period of time, and then (3) active for a predetermined period of time, the multi-stage MEOI detection algorithm is configured to minimize the amount of time that the medical device 12 performs the frequency analysis, thereby reducing the overall computational requirements and battery consumption of the medical device 12.

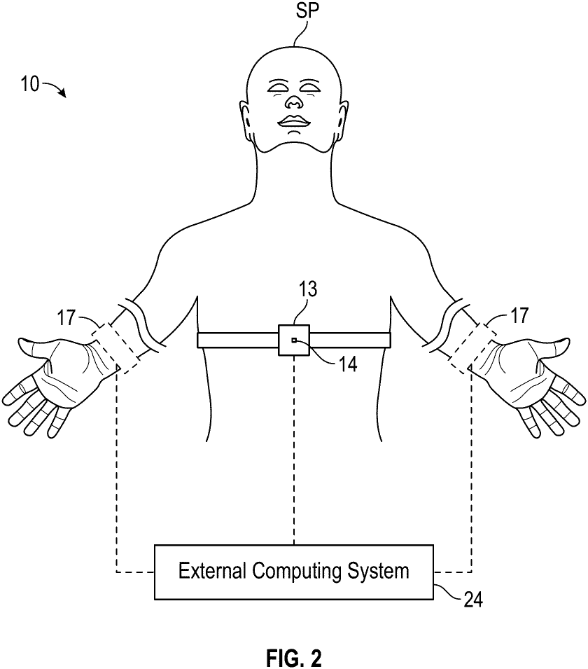

As shown in FIG. 1, the medical device 12 is implanted within a torso or chest 16 of the subject patient SP. For example, in some instances, the medical device 12 may be an implantable stimulation device including the one or more acceleration sensors 14 and an electrically conductive lead assembly 18 configured to transmit stimulation signals to one or more nerve electrodes 20 (or alternatively 20′). In these instances, the one or more nerve electrodes 20 may be surgically coupled to and configured to deliver stimulation to a nerve of the subject patient SP (e.g., a vagus nerve 22 or alternatively 22′). However, it should be appreciated that, in some instances, the medical device 12 may alternatively comprise an implantable medical device including the one or more acceleration sensors 14 without stimulation capabilities. In yet some other instances, the medical device 12 may be an external medical device (e.g., external medical device 13 shown in FIG. 2) that is wearable on the chest 16 of the subject patient SP. Further, in some instances, the system 10 may optionally additionally or alternatively include one or more limb-based (e.g., wrist-based, leg-based) medical devices 17. In some instances, the seizure detection system 10 may comprise both implantable and external medical devices (e.g., medical device 12 and external medical device 13) to provide beneficial aspects of both the implantable medical devices (e.g., responsive stimulation capabilities, accurate device location and orientation) and external medical devices (e.g., convenient recharging, patient notification capabilities).

The medical device 12 may be programmed using an external computing system 24 (e.g., a computer, tablet, smart phone). In some instances, a programming wand 26 may be coupled to the external computing system 24 (e.g., via a wired or wireless connection) to facilitate radio frequency (RF) communication between the external computing system 24 and the medical device 12. In some other instances, the external computing system 24 may be configured to communicate directly with the medical device 12 using close-range wireless communication signals (e.g., Bluetooth, Wi-Fi).

Accordingly, the programming wand 26 and/or the external computing system 24 may be configured to permit non-invasive communication with the medical device 12 after the latter is implanted. In systems where the external computing system 24 is configured to use close-range wireless communication signals to communicate directly with the medical device 12, the programming wand 26 may be omitted. Further, in systems alternatively including the external medical device 13, the external computing system 24 and/or the programming wand 26 may be configured to communicate with the external medical device 13 via wired or wireless connections to program the external medical device 13.

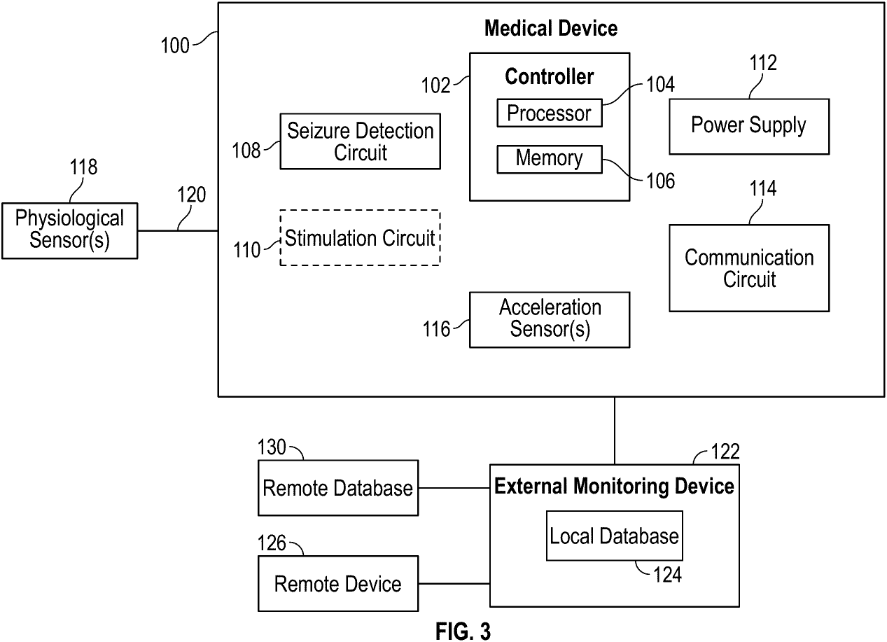

Turning now to FIG. 3, a block diagram depiction of a medical device 100 is provided, according to an exemplary embodiment. It should be appreciated that the medical device 100 may be substantially similar to any of the medical devices 12, 13, 17 shown in FIGS. 1 and 2. As such, it will be appreciated that the following description of medical device 100 may similarly be applied to any of the medical devices 12, 13, 17 described above.

As shown in FIG. 3, in some embodiments, the medical device 100 comprises a controller 102 capable of controlling various aspects of the operation of the medical device 100. The controller 102 comprises a processor 104 and a memory 106. The processor 104 may comprise one or more microcontrollers, microprocessors, etc., capable of performing various executions of software components. The memory 106 may comprise various memory portions where a number of types of data (e.g., internal data, external data instructions, software codes, status data, diagnostic data, etc.) may be stored. The memory 106 may comprise one or more of random access memory (RAM), dynamic random access memory (DRAM), electrically erasable programmable read-only memory (EEPROM), flash memory, etc.

The controller 102 is configured to receive internal data and/or external data and to detect, via a seizure detection circuit 108, whether the patient is experiencing a seizure event. For example, the controller 102 is configured to detect whether the patient is experiencing a generalized tonic-clonic, focal to bilateral tonic-clonic, and/or clonic seizure events, as will be described in detail below. The seizure detection circuit 108 may comprise various software module(s) that are capable of performing various interface functions, frequency analysis functions, etc., to perform the various methods described herein. In another embodiment the seizure detection circuit 108 may comprise hardware circuitry that is capable of performing these functions. In yet another embodiment, the seizure detection circuit 108 may comprise hardware, firmware, software and/or any combination thereof.

In some instances, in response to a detected seizure event, via the seizure detection circuit 108, the controller 102 may optionally be further configured to cause a stimulation circuit 110 to generate and deliver an electrical signal to target tissues of the patient’s body (e.g., via the electrically conductive lead assembly 18 and nerve electrodes 20 (or 20′) shown in FIG. 1) for treating and/or counteracting the seizure event. In these instances, the therapy may be delivered to the target tissues by the stimulation circuit 110 based upon instructions from the controller 102.

The stimulation circuit 110 may comprise various circuitry, such as electrical signal generators, impedance control circuitry to control the impedance “seen” by the leads, and other circuitry that receives instructions relating to the delivery of the electrical signal to tissue. However, in some other embodiments, the medical device 100 may not comprise a stimulation circuit 110 (nor the corresponding lead assembly 18 or electrodes 20 (or 20′) shown in FIG. 1). In any case, the controller 102 is capable of affecting substantially all functions of the medical device 100.

The medical device 100 further comprises a power supply 112. The power supply 112 may comprise a battery, voltage regulators, capacitors, etc., to provide power for the operation of the medical device 100, including monitoring of the patient’s posture and movement activity, performing frequency analyses, and, in some instances, delivering a therapeutic electrical signal.

The power supply 112 comprises a power source that in some embodiments may be rechargeable. In other embodiments, a non-rechargeable power source may be used. The power supply 112 provides power for the operation of the medical device 100, including electronic operations and, in some instances, the electrical signal generation and delivery functions.

The medical device 100 further comprises a communication circuit 114 capable of facilitating communications between the medical device 100 and various external devices. In particular, the communication circuit 114 is capable of providing transmission and reception of electronic signals to and from the various external devices. The communication circuit 114 may thus include associated hardware, software, firmware, or any combination thereof for facilitating these communications.

The medical device 100 further comprises one or more acceleration sensor(s) 116 (e.g., similar to the acceleration sensor 14 shown in FIGS. 1 and 2). The one or more acceleration sensor(s) 116 are configured to receive signals related to the patient’s posture and movement. In some instances, the one or more acceleration sensor(s) 116 comprise a single three-directional acceleration sensor configured to sense acceleration in three orthogonal directions. In some other instances, the one or more acceleration sensor(s) 116 may comprise three single-directional acceleration sensors arranges to sense acceleration in three orthogonal directions. In yet some other instances, the one or more acceleration sensor(s) 116 may comprise more or less than three single-direction or one multi-directional acceleration sensors arranged in various manners, as desired for a given application.

In some instances, the medical device 100 may optionally further comprise one or more physiological sensor(s) 118 coupled via sensor lead(s) 120 to the medical device 100. The physiological sensor(s) 118 may be configured to receive signals related to various physiological parameters, such as the patient’s heart rate, blood pressure, respiration rate, perspiration level, and/or temperature, and deliver the signals to the medical device 100. In one embodiment, the physiological sensor(s) 118 may be implanted electrode(s) (e.g., similar to or the same as nerve electrodes 20 shown in FIG. 1). In other embodiments, the sensor(s) 118 are external structures that may be placed on the patient’s skin, such as over the patient’s heart or elsewhere on the patient’s torso.

In some embodiments, the memory 106 of the medical device 100 is configured to store indications of detected seizure events. In some embodiments, the medical device 100 may be communicatively coupled to an external monitoring device 122 (e.g., similar to the external computing system 24 shown in FIGS. 1 and 2) comprising a local database 124 configured to store the indications of detected seizure events. In these instances, the medical device 100 may communicate the indications to the external monitoring device 122 via the communication circuit 114. In some instances, the medical device 100 may communicate these indications in real-time to the external monitoring device 122, which may further communicate the indication to one or more remote device(s) 126 associated with caregivers and/or healthcare professionals to provide an alert that the patient is currently experiencing a seizure event.

The external monitoring device 122 may be a device that is capable of transmitting and receiving data to and from the medical device 100. In one embodiment, the monitoring device 122 is a computer system capable of executing a data-acquisition program (e.g., similar to the external computing system 24 shown in FIGS. 1 and 2). In some embodiments, the monitoring device 122 may be configured for use by a healthcare provider, such as a physician, at a base station in, for example, a doctor’s office. In other embodiments, the external monitoring device 122 may be configured for use by a patient.

For example, in some instances a first monitoring device may be used by a physician to program the medical device 100 and a second monitoring device may be used by the patient at home to allow for the patient to provide various inputs, such as, for example, indications that the patient is about to lay down to sleep, that the patient is getting up from sleeping, that the patient has or has not experienced a seizure event, etc.

Whether controlled by the patient or by a healthcare provider, the monitoring device 122 may be any suitable computing device that is capable of electronic communications and programming, e.g., a desktop computer, a laptop, a smart phone, a tablet, a server, a personal digital assistant (PDA), etc. The monitoring device 122 may upload various parameters and program software into the medical device 100 for programming the operation of the medical device, and may also receive and download various patient status conditions and other data from the medical device 100.

Communications between the monitoring device 122 and the communication circuit 114 in the medical device 100 may occur via a wireless or other type of communication. This may occur using, e.g., a programming wand (e.g., the wand 26 shown in FIG. 1) to communicate by RF energy with the medical device 100. Alternatively, the wand may be omitted in some systems in which the medical device 100 is non-implantable or implantable systems in which monitoring device 122 and medical device 100 utilize wireless communication forms.

In one embodiment, the monitoring device 122 may comprise the local database 124. Additionally or alternatively, the monitoring device 122 may also optionally be coupled to a remote database 130, which may be separate from monitoring device 122 (e.g., a centralized database wirelessly linked to the external monitoring device 122). The local database 124 and/or the remote database 130 may be capable of storing various patient data. These data may comprise patient parameter data acquired from the patient’s body (e.g., patient posture and movement data, other physiological data, seizure severity data, therapeutic efficacy data) and/or stimulation therapy parameter data.

In some instances, the local database 124 and/or the remote database 130 may comprise data for a plurality of patients. The data for the plurality of patients may be organized and stored in a variety of manners, such as in date format, severity of disease format, etc.

In some instances, a physician may be allowed to perform various patient management functions using the monitoring device 122, which may include obtaining and/or analyzing data from the medical device 100 and/or data from the local database 124 and/or the remote database 130, setting thresholds for one or more detection parameters, and/or programming parameters for a responsive stimulation therapy.

It should be appreciated that one or more of the blocks illustrated in the block diagram of the medical device 100 shown in FIG. 3 may comprise hardware units, software units, firmware units, or any combination thereof. Additionally, one or more blocks illustrated in FIG. 3 may be combined with other blocks. Additionally, in some instances, any number of the circuitry or software units associated with the various blocks illustrated in FIG. 3 may be combined into a programmable device, such as a field programmable gate array, an ASIC device, etc.

Now that the various components of the system 10 and the medical device 100 have been described above, a method for utilizing the multi-stage MEOI detection algorithm to detect patient seizure events will be described below. It will be appreciated that the following method is provided as an example and, as such, various modifications, omissions, and additions are contemplated by and within the scope of the present disclosure. Furthermore, although the following description will be provided in reference to medical device 100, it may similarly be applied to any of the other medical devices described herein (e.g., medical device 12, medical device 13).

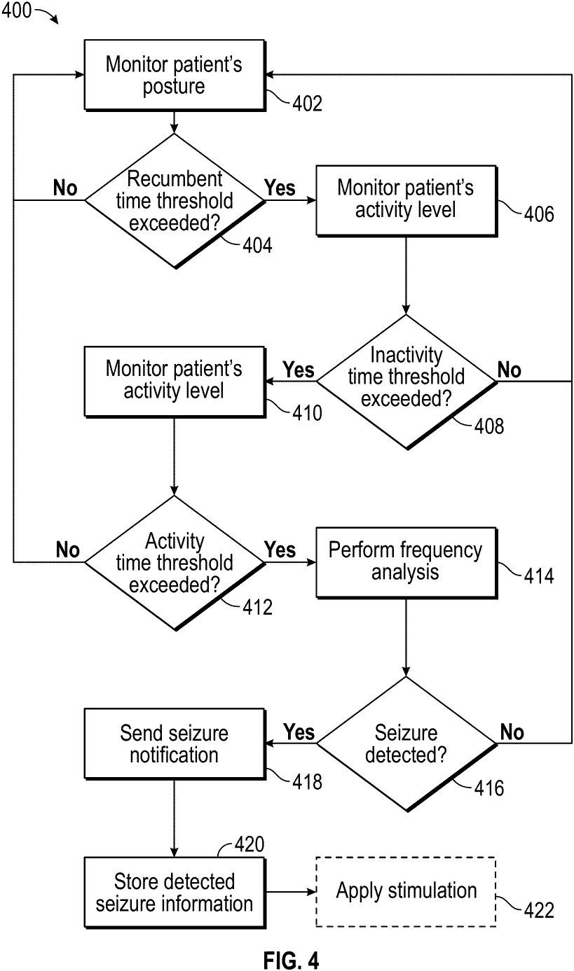

Referring to FIG. 4, a method 400 for detecting seizure events utilizing a multi-stage movement event of interest (MEOI) detection algorithm by the medical device 100 is shown, according to an exemplary embodiment. The multi-stage MEOI detection algorithm is configured to reliably detect movements that are not physiological (e.g., seizure events) during periods where the patient is lying down at rest (e.g., when the patient is asleep) and when the patient’s movements have satisfied various relevant criteria, as will be discussed in detail below.

For example, during use, the seizure detection circuit 108 begins by monitoring the patient’s posture, at step 402. The seizure detection circuit 108 is configured to monitor the patient’s posture using posture data received from the acceleration sensor(s) 116, which may be located in the torso or chest 16 of the subject patient SP. In some embodiments, the patient’s posture is continuously assessed. In other embodiments, the patient’s posture may only be assessed during pre-specified times throughout the day (e.g., at nighttime when the patient is more likely to be sleeping). The pre-specified times may be selected by the patient, a caretaker, and/or a healthcare provider via the external computing system 24, the external monitoring device 122, and/or the remote device 126 and communicated to the medical device 100. In some other embodiments, the monitoring of the patient’s posture may be triggered by an input from the patient. For example, the patient may provide an input indicating that the patient is laying down to sleep, which may trigger the medical device 100 to begin monitoring the patient’s posture.

While monitoring the patient’s posture, the seizure detection circuit 108 is then configured to determine whether the patient has been in a recumbent position for longer than a recumbent time period threshold, at step 404. For example, in some instances, the recumbent time period threshold may be pre-programmed into the medical device 100. In other embodiments, the recumbent time period threshold may be selected by a healthcare professional via any of the external computing system 24, the external monitoring device 122, and the remote device 126. For example, in some instances, the recumbent time period threshold may be set to 3 or 4 seconds. In some instances, the recumbent time period threshold may be set to between 1 and 10 seconds. In any case, this determination of whether the patient has been recumbent for longer than the recumbent time period threshold may be referred to as the recumbent posture gate.

If the seizure detection circuit 108 determines that the recumbent time period threshold has not been exceeded, at step 404, the seizure detection circuit 108 then returns to monitoring the patient’s posture, at step 402. Alternatively, if the seizure detection circuit 108 determines that the recumbent time period threshold has been exceeded, at step 404, the seizure detection circuit 108 may then begin to monitor the patient’s activity level, at step 406, using the acceleration information received from the acceleration sensors 116.

The seizure detection circuit 108 is then configured to determine whether the patient has been inactive for longer than an inactivity time period threshold, at step 408. In some instances, this determination of whether the patient has been inactive for longer than the inactivity time period threshold may be referred to as the patient inactivity gate. In any case, to determine whether the patient is inactive at any given moment, the seizure detection circuit 108 may compare the patient’s activity level (e.g., a magnitude of the patient’s movement deduced from the acceleration information) to a first patient activity threshold.

In some instances, the inactivity time period threshold and/or the first patient activity threshold may be pre-programmed into the medical device 100. However, in some other instances, it may be beneficial to incorporate a patient-specific adjustment of the first patient activity threshold to allow for a more accurate seizure detection process. For example, the patient’s activity level (e.g., the magnitude of the patient’s movement) may vary as a function of the patient’s general activity level (e.g., their physical fitness level), body mass, and/or the material/nature of the surface they are situated on. Accordingly, in these instances, the inactivity time period threshold and/or the first patient activity threshold may be selected by a healthcare professional via any of the external computing system 24, the external monitoring device 122, and the remote device 126. For example, in some instances, the healthcare professional may be allowed to select the inactivity time period threshold and/or the first patient activity threshold from multiple predetermined settings (e.g., high, medium, low). In some instances, the inactivity time period threshold may be set to 10 or 12 seconds. In other instances, the inactivity time period threshold may be set between 5 and 20 seconds. In some instances, the first patient activity threshold (e.g., a quiet threshold) may be set to approximately 0.035 G, such that activity below 0.035 G is treated as “quiet” or “inactive.”

If the seizure detection circuit 108 determines that the patient has not been inactive for longer than the inactivity time period threshold within a first predetermined amount of time (e.g., a first time out window), the seizure detection circuit 108 returns to monitoring the patient’s posture, at step 402. Alternatively, if the seizure detection circuit 108 determines that the patient has been inactive for longer than the inactivity time period threshold, the seizure detection circuit 108 then continues to monitor the patient’s activity level, at step 410.

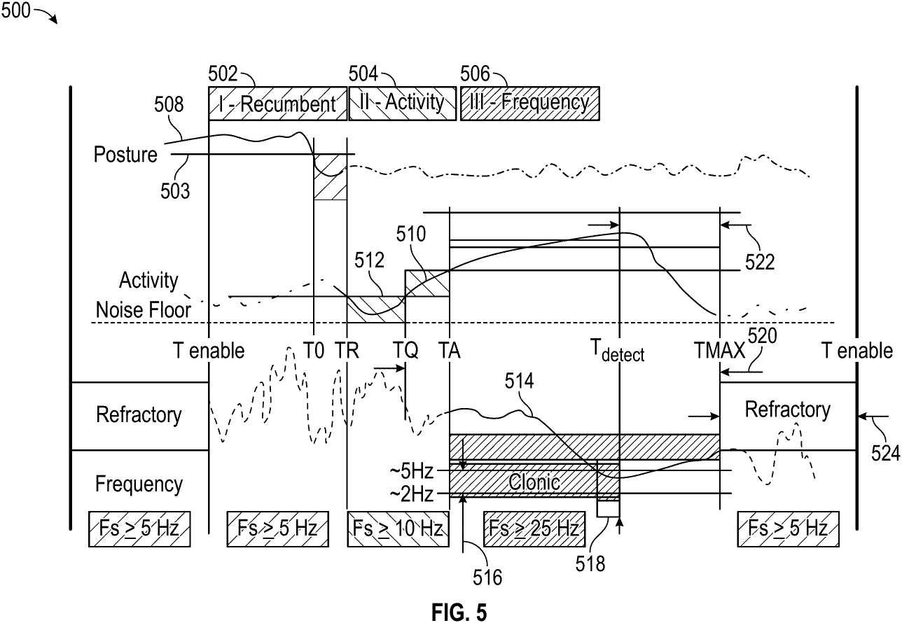

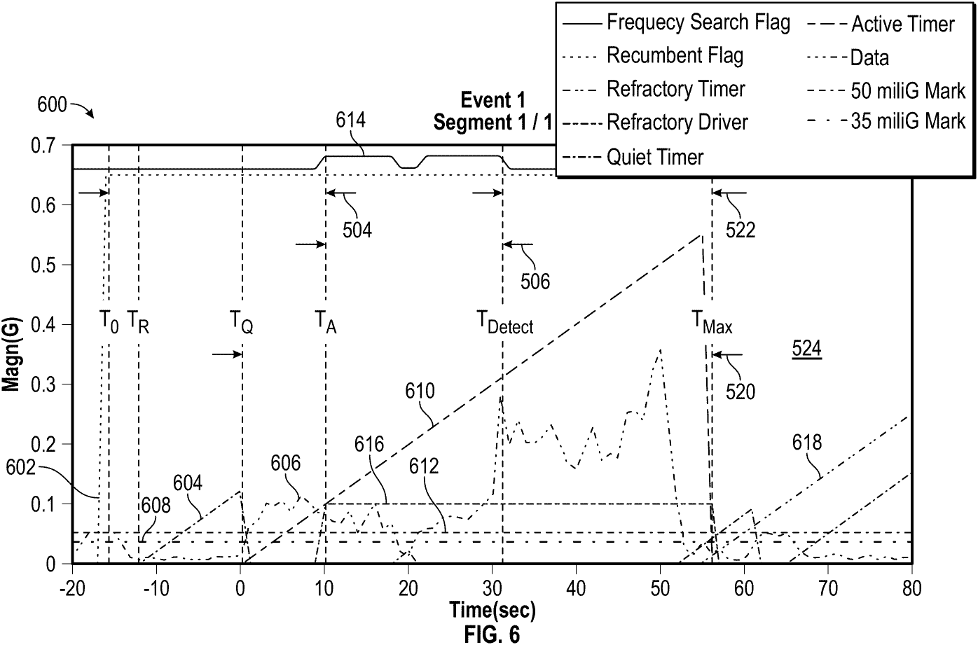

At this point, the seizure detection circuit 108 determines whether, after the patient has been inactive for longer than the inactivity time period threshold, the patient has subsequently been active for longer than an activity time period threshold, at step 412. In some instances, the determination of whether the patient has been active for longer than the activity time period threshold may be referred to as the patient activity gate. To determine whether the patient is active at any given moment, the seizure detection circuit 108 may similarly compare the patient’s activity level to a second patient activity threshold. In some instances, the first patient activity threshold and the second patient activity threshold may be the same threshold (as shown in FIG. 5). In other instances, the first patient inactivity threshold and the second patient activity threshold may be different thresholds (as shown in FIG. 6).

Similarly, in some instances, the activity time period threshold and/or the second patient activity threshold may be pre-programmed into the medical device 100 or selected by a healthcare professional via any of the external computing system 24, the external monitoring device 122, and the remote device 126. For example, in some instances, the healthcare professional may similarly be allowed to select the activity time period threshold and/or the second patient activity threshold from multiple predetermined settings (e.g., high, medium, low). In some instances, the activity time period threshold may similarly be set to 10 or 12 seconds. In other instances, the activity time period threshold may similarly be set between 5 and 20 seconds. In some instances, the second patient activity threshold (e.g., an active threshold) may be set to approximately 0.050 G, such that activity above 0.050 G is treated as “active.”

In some instances, in addition to the first and second patient activity thresholds (e.g., the quiet and active thresholds), the system may further be configured to compare the patient’s activity to a third patient activity threshold, above which the patient’s activity may be treated as a non-seizure condition. In these instances, the third patient activity threshold may be set higher than both the first and second patient activity thresholds.

In some instances, the method 400 may allow for the patient activity level to drop below the patient activity threshold momentarily while still considering the patient “active” if the patient activity level rises back above the patient activity threshold within a predetermined grace period. The predetermined grace period may similarly be pre-programmed into the medical device or selected by the healthcare professional via any of the external computing system 24, the external monitoring device 122, and the remote device 126.

If the seizure detection circuit 108 determines that the patient has not been active for longer than the activity time period threshold within a second predetermined amount of time (e.g., a second time out window), the seizure detection circuit 108 may return to monitoring the patient’s posture, at step 402. It should be appreciated that, if patient has remained in a recumbent position, the medical device 100 may instantly proceed through monitoring the patient’s posture, at step 402, and determining whether the recumbent time period threshold has been exceeded, at step 404, and immediately begin monitoring the patient’s activity level at step 406 and determining whether the inactivity time period threshold has been exceeded, at step 408.

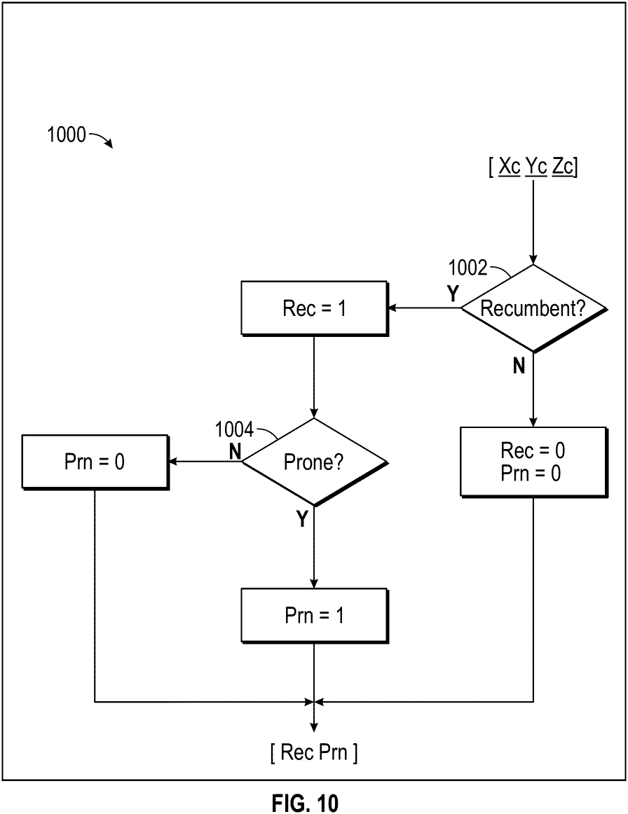

Alternatively, if the seizure detection circuit 108 determines that the patient has been active for longer than the activity time period threshold, the medical device 100 may then begin to perform a frequency analysis of the patient’s movement data, at step 414, and determine whether the patient is experiencing a seizure event, at step 416. Specifically, in some instances, a specialized radix-2 real decimation in time (DIT) fast Fourier transform (FFT) analysis is applied to obtained X, Y, and/or Z data from the acceleration sensor 116 to determine the fundamental frequency of the patient’s movements. This specialized radix-2 real DIT FFT analysis is described in detail below, with respect to FIGS. 11-17. In some other instances, traditional discrete Fourier transform and/or FFT analyses may be applied to the obtained X, Y, and/or Z data to determine the fundamental frequency of the patient’s movements.

In any case, the seizure detection circuit 108 then determines whether the fundamental frequency of the patient’s movements falls within a narrowband of frequencies corresponding to the typical frequency band associated with seizure events for a minimum seizure detection period. For example, typical physiological movements often have higher acceleration. However, during sleep, the expected acceleration of the patient’s movements is lower. Typical frequency of movement during physiological activity are in the range of <2 Hz for slow walks, brushing of teeth, and between 4-6 Hz for fast walking and running. Seizures such as generalized tonic-colonic seizures generally occur within a convulsion frequency band of 2-5 Hz and clonic seizures generally occur within a frequency band of 1-3 Hz. It has been shown that such (i.e., convulsive) movements will have similar acceleration and frequency levels on the torso/trunk and arms however the amplitudes will be somewhat different.

Accordingly, the seizure detection circuit 108 may compare the fundamental frequency of the patient’s movement detected via the acceleration sensor 116 (e.g., within the medical device 100 implanted in the patient’s chest/torso and/or worn on the patient’s chest and/or limb(s)) to the narrowband of frequencies corresponding to the typical frequency band of generalized tonic clonic, tonic-clonic, and/or clonic seizures to determine whether the patient is experiencing a seizure event. That is, if the seizure detection circuit 108 determines that the frequency of the patient’s movement falls within the narrowband of frequencies for the minimum seizure detection period, the seizure detection circuit 108 may then determine that the patient is experiencing a generalized tonic-clonic, a tonic-clonic, or a clonic seizure.

It should be appreciated that if, while performing the frequency analysis, at step 414, and/or while determining whether a seizure has been detected, at step 416, the patient’s fundamental frequency strays outside of the required narrowband of frequencies, the seizure detection circuit 108 may reset a seizure detection timer and continue analyzing and monitoring the patient’s fundamental frequency. The frequency analysis continues until either the required minimum seizure detection period is reached or the frequency analysis times out.

In some instances, the minimum seizure detection period may be a preset time period programmed into the medical device. In other instances, the minimum seizure detection period may be set and/or adjusted by a healthcare professional via the external computing system 24, the external monitoring device 122, and/or the remote device 126.

It should be appreciated that, in some instances, a system (e.g., similar to the system 10) may utilize a limb-based (e.g., wrist-based) medical device. In these instances, because limbs may be in random orientations while the patient is in the recumbent position, the multi-stage MEOI algorithm may be adapted to skip the posture-related steps (i.e., step 402 and step 404) and proceed directly to step 406 of the method 400.

It should further be appreciated that, while chest-based or limb-based acceleration data may be utilized to determine the fundamental frequency of the patient’s movements, chest-based acceleration data (e.g., obtained via acceleration sensors implanted within or worn on the patient’s chest) may provide a more accurate fundamental frequency of the patient’s movements as compared to limb-based acceleration data (e.g., obtained via limb-based wearable medical devices, similar to the medical device 17 shown in FIGS. 1 and 2).

However, in some instances, chest-based and limb-based medical devices (e.g., implanted or wearable) may both be utilized in a single system to provide a multi-device analysis to allow for more accurate seizure event detection. Additionally, in some instances, wearable medical devices (e.g., medical devices 13, 17) may be rechargeable, whereas implanted medical devices (e.g., medical device 12) may either not be rechargeable or may be more difficult to recharge. Accordingly, in these instances, systems utilizing both implanted medical devices and wearable medical devices may beneficially ensure continuous data collection and analysis by allowing for the rechargeable wearable medical devices to be utilized in the case that the implantable medical device runs out of power. However, it should be appreciated that either of the implanted medical devices and/or the wearable medical devices may have one or more rechargeable batteries and/or other power sources configured to be recharged during use and/or between uses.

In some instances, while monitoring the patient’s activity level, at steps 406 and 410, determining whether the patient has been inactive and/or active for longer than the inactivity time period threshold and/or the activity time period threshold, at steps 408 and 412, performing the frequency analysis, at step 414, and determining whether the patient is experiencing a seizure, at step 416, the medical device 100 is configured to continuously monitor the patient’s posture. If, at any time, the patient is no longer recumbent, the medical device 100 is configured to return to monitoring the patient’s posture, at step 402, and determining whether the patient has again been recumbent for longer than the recumbent time period threshold, at step 404.

Similarly, while performing the frequency analysis, at step 414, and determining whether the patient is experiencing a seizure, at step 416, the medical device 100 is configured to continuously monitor the patient’s activity level. If, at any time, the patient’s activity level drops below the patient activity threshold, the medical device 100 is configured to stop performing the frequency analysis until the patient’s activity level rises back above the patient activity threshold. However, if the patient’s activity level drops below the patient activity threshold for longer than a predetermined grace period, the medical device 100 may be configured to return to monitoring the patient’s activity level, at step 410, and determining whether the patient has been active for longer than the activity time period threshold, at step 412.

If the seizure detection circuit 108 does not determine that the patient is experiencing a seizure event within the minimum seizure detection period, at step 416, the seizure detection circuit 108 may then return to monitoring the patient’s posture, at step 402, and determining whether the recumbent time period threshold has been exceeded, at step 404.

Alternatively, if the seizure detection circuit 108 determines that the patient is experiencing a seizure event, at step 416, the seizure detection circuit 108 may then send a notification that the patient is or may be experiencing a seizure, at step 418, to any of the external computing system 24, the external monitoring device 122, and/or the remote device 126 via the communication circuit 114. In this way, the patient, a care giver, and/or a medical professional may be alerted that the patient is experiencing a potential tonic-clonic seizure.

For example, in some instances, if the seizure detection circuit 108 determines that the patient is experiencing a seizure event, at step 416, and the seizure detection system (e.g., seizure detection system 10) includes an external medical device (e.g., the external medical device 13 in conjunction with an implantable medical device or by itself), the seizure detection circuit 108 may send a notification to the external medical device, which may include a display configured to provide a visual alert to the patient, a speaker or other audio device configured to provide an audible alert to the patient, a tactile alert mechanism (e.g., a vibration mechanism or vibrator) configured to provide a tactile alert to the patient, or any other suitable alert device configured to alert the patient that they are experiencing or have experienced a potential seizure event. The seizure detection circuit 108 may then also store the detected seizure information in the memory 106 and/or send the detected seizure information to an external device (e.g., the external computing system 24, the external monitoring device 122, and/or the remote device 126) to be stored, at step 420.

In some instances, upon determining that the patient is experiencing a seizure event, at step 416, the seizure detection circuit 108 may communicate this information to the controller 102, which may in turn control the stimulation circuit 110 to apply anti-seizure stimulation therapy to the patient, at step 422, to counteract the patient’s seizure.

In some instances, upon determining that the patient is experiencing a seizure event based on the acceleration data using the multi-stage MEOI detection algorithm utilized in the method 400, as described above, the medical device 100 may then be configured to perform a secondary seizure detection determination utilizing various physiological signals obtained by the one or more physiological sensors 118 to provide a detection confirmation stage to further improve accuracy of the seizure detection capabilities of the medical device 100.

For example, in some instances, the medical device 100 may be configured to detect seizure events based on the patient’s perspiration level, the patient’s respiration rate, and/or cardiac signals. In some instances, detection of seizure events based on the acceleration data using the multi-stage MEOI detection algorithm utilized in the method 400 may precede possible detection of the seizure events based on other physiological signals. For example, detection of seizure events based on the acceleration data may precede possible detection of the seizure events based on cardiac signals. That is, during a seizure event, the patient may begin to show motion-based indications indicative of a seizure event prior to the patient’s cardiac signal (e.g., heart rate) beginning to show indications indicative of the seizure event.

It should be appreciated that the multi-stage MEOI detection algorithm utilized in the method 400 allows for the frequency of the patient’s movements to be analyzed only at specific times where the patient’s movement data meets each of the criteria specified above. That is, prior to performing the frequency analysis, the patient must first be recumbent for a first period of time, then continuously inactive for a second period of time, then continuously active for a third period of time. This particular order of thresholds or gates must be met before the medical device 100 triggers the frequency analysis. Further, throughout the process after the patient is recumbent for the first period of time, the medical device 100 further continuously monitors the patient’s posture, and if, at any time, the patient is no longer recumbent, the medical device 100 will not perform the frequency analysis. Additionally, while performing the frequency analysis after the patient has been continuously active for the third period of time, if the patient becomes inactive (i.e., the patient’s activity level drops below the patient activity threshold), the medical device 100 is configured to stop the frequency analysis.

Accordingly, the multi-stage MEOI detection algorithm utilized in the method 400 is configured to prevent the medical device 100 from performing the frequency analysis except when the appropriate seizure precursor event is first identified (i.e., when the specific criteria related to posture, inactivity, and activity discussed above are met), thereby minimizing the fraction of the operation time the medical device 100 spends performing the frequency analysis and reducing the overall computational requirements and associated power consumption of the medical device 100.

It should further be appreciated that it is also beneficial for avoiding false positive detections to ensure that all of the criteria of the multi-stage MEOI detection algorithm is satisfied prior to performing the frequency analysis, since normal patient activity (i.e., non-seizure-event activity) may result in motion within the frequency band of interest (e.g., the clonic frequency band).

In some instances, the medical device 100 may further be configured to detect when the patient experiences a fall event based on the acceleration data obtained by the acceleration sensor 116. In these instances, the medical device 100 may utilize the detected fall event as an overriding seizure precursor event within the multi-stage MEOI algorithm discussed above to bypass the posture, inactivity, and/or activity monitoring and determination steps in the method 400. For example, in some instances, upon detecting a patient fall event, the multi-stage MEOI algorithm may instantly begin to perform the frequency analysis, at step 414. In some other instances, upon detecting a patient fall event, the multi-stage MEOI algorithm may instead instantly start monitoring the patient’s activity level, at step 410.

It should be appreciated that a variety of seizure precursor events may be additionally and/or alternatively utilized within the multi-stage MEOI algorithm to trigger the frequency analysis, at step 414. For example, in some instances, various physiological parameters of the patient may be utilized to identify a variety of seizure precursor events. That is, in some instances, various physiological parameters, such as, for example, the patient’s heart rate, blood pressure, respiration rate, perspiration level, and/or temperature may be utilized to identify a variety of seizure precursor events.

For example, in some instances, a seizure precursor event may be identified based on a sudden change in one or more of the patient’s heart rate, blood pressure, respiration rate, perspiration level, temperature, and/or any other relevant physiological parameter of the patient. In any case, upon detection of any of the seizure precursor events described herein, the multi-stage MEOI algorithm may bypass one or more of the posture, inactivity, and/or active monitoring and determination steps in the method 400 and/or may instantly begin to perform the frequency analysis, at step 414.

For example, when the patient experiences a seizure event while they are standing, they will generally fall to the ground. Accordingly, in the instances where the medical device 100 is configured to detect patient fall event, the medical device 100 may still be configured to detect the seizure event experienced by the patient even though they were not recumbent for the recumbent time period threshold and inactive for the inactivity time period threshold.

In some instances, the medical device 100 may be further configured to receive feedback from the patient regarding experienced seizure events (e.g., via any of the external computing system 24, the external monitoring device 122, and/or the remote device 126). In these instances, the medical device 100 may be configured to automatically update any of the recumbent time period threshold, the inactivity time period threshold, the activity time period threshold, the patient activity threshold, and/or the minimum seizure detection period based on the feedback from the patient.

For example, if the patient indicates that they experienced a seizure event, but no seizure event was detected, this may indicate to the medical device 100 that various thresholds and/or the minimum seizure detection period should be reduced. Conversely, if the medical device 100 detects a seizure event, but the patient indicates that no seizure event was experienced, this may indicate to the medical device that various thresholds and/or the minimum seizure detection period should be increased. In some instances, the medical device 100 may be configured to modify the various thresholds and/or the minimum seizure detection period only after a predetermined number of false positive seizure detections or failures to detect experienced seizure events.

Referring now to FIG. 5, an exemplary plot 500 is shown illustrating the patient’s posture, activity level, and movement frequency throughout the method 400 of detecting a seizure event during operation of the medical device 100, according to an exemplary embodiment. As illustrated in FIG. 5, throughout the method 400, the medical device 100 may enter a recumbent mode 502, an activity mode 504, and a frequency mode 506, in which the seizure detection circuit 108 is configured to monitor the patient’s posture, monitor the patient’s activity level, and perform a frequency analysis of the patient’s movements, respectively. As illustrated, the sampling frequency Fₛ may have varying levels from mode to mode. For example, in the recumbent mode 502, the sampling frequency Fₛ may be greater than or equal to 5 Hz. In the activity mode 504, the sampling frequency Fₛ may be greater than or equal to 10 Hz. In the frequency mode 506, the sampling frequency Fₛ may be greater than or equal to 25 Hz.

The recumbent mode 502 starts as soon as the medical device 100 is enabled at T_{enable}. As shown, at a first time T₀, a posture signal 508 shows the patient’s posture changing from an upright position to a recumbent position (i.e., falling below a recumbent position threshold 503). The posture signal 508 is detected by the seizure detection circuit 108 using the acceleration data received from the acceleration sensors 116. After the patient has been recumbent for longer than the recumbent time period threshold, at time T_{R}, the seizure detection circuit 108 is triggered to switch into the activity mode 504.

As shown, during the activity mode 504, a patient activity signal 510 shows the patient’s activity level dropping below a patient activity threshold 512 at time T_{R}. The patient activity signal 510 is similarly detected by the seizure detection circuit 108 using the acceleration data received from the acceleration sensors 116. After the patient has been inactive for the inactivity time period threshold at time T_{Q}, the patient’s activity level then rises above the patient activity threshold 512, at time T_{Q}. As discussed above, in some instances, the patient activity threshold may for the inactive and active determinations may be the same threshold (e.g., the patient activity threshold 512). However, it should be appreciated that, in some instances, the patient activity threshold for the inactive and active determinations may be different.

In any case, after the patient has been active for the activity time period threshold, at time T_{A}, the medical device 100 is triggered to switch into the frequency mode 506. During the frequency mode 506, a frequency signal 514 shows the frequency of the patient’s movements falling down into a clonic frequency narrowband 516. Once the frequency of the patient’s movements has remained within the clonic frequency narrowband 516 for a minimum seizure detection period 518, the seizure detection circuit 108 detects that the patient is experiencing a seizure, at time T_{detect.}

In some instances, the medical device 100 may have a maximum time-out period 520, shown in FIG. 5 as the time period between T_{Q} and T_{MAX}. That is, after the patient has been inactive for the inactivity time period threshold, the medical device 100 may be configured to subsequently look for the patient’s activity rising above the patient activity threshold 512 and perform the frequency analysis to detect seizure events within the maximum time-out period 520.

In any case, once the frequency mode 506 is triggered, at time T_{A}, the seizure detection circuit 108 is configured to perform the frequency analysis of the patient’s movements until the maximum time-out period 520 has been reached. In some instances, if, as shown in FIG. 5, a seizure is detected before the maximum time-out period 520 has ended, the seizure detection circuit 108 may then enter an interim period 522 until the end of the maximum time-out period 520 and subsequently enter a refractory period 524. In some other instances, if a seizure is detected within the maximum time-out period 520, the seizure detection circuit 108 may be configured to instantly enter the refractory period 524. In any case, after the refractory period 524 or if a seizure is not detected within the maximum time-out period 520, the seizure detection circuit 108 may be configured to restart the multi-stage MEOI detection algorithm by re-entering the recumbent mode 502.

During the refractory period 524, the medical device 100 may be configured to temporarily suspend all monitoring of the patient. The medical device 100 may then restart the method 400 after completion of the refractory period 524. In some instances, the refractory period 524 may be pre-programmed into the medical device 100. In other instances, the refractory period 524 may be selected by a healthcare professional, a caretaker, or the patient via the external computing system 24, the external monitoring device 122, and/or the remote device 126. In yet some other instances, the medical device 100 may not enter a refractory period 524 at all, and may immediately begin to monitoring the patient’s posture after the seizure is detected or the maximum time-out period 520 has expired.

Referring now to FIG. 6, a plot 600 showing the patient’s activity level (shown by patient activity signal 606) through a portion of the recumbent mode 502, the activity mode 504 and frequency mode 506 and associated timers involved in the multi-stage MEOI detection algorithm, according to an exemplary embodiment. For example, a recumbent flag 602 is initially triggered, at time T₀, by the patient’s posture indicating the patient is in the recumbent position.

The medical device 100 then enters the activity mode 504 and a patient inactivity timer 604 begins when the patient activity signal 606 drops below a first patient activity threshold 608 after the patient has been in the recumbent position for longer than the recumbent time period threshold, at time T_{R}. As illustrated, the patient remains in the recumbent position throughout the activity mode 504, the frequency mode 506, the interim period 522, and into the refractory period 524. The patient inactivity timer 604 continues as long as the patient activity signal 606 remains below the first patient activity threshold 608 and until the patient activity signal 606 has been below the first patient activity threshold 608 for the inactivity time period threshold, at time T_{Q}. After the patient activity signal 606 has remained below the first patient activity threshold 608 for the inactivity time period threshold, at time T_{Q}, a patient activity timer 610 is triggered.

The patient activity timer 610 may continue for the duration of the maximum time-out period 520 (e.g., the time period between T_{Q} and T_{Max}). While the patient activity timer 610 is running, the medical device 100 monitors the patient activity level and continuously compares the patient’s activity level to the first patient activity threshold 608 and/or a second patient activity threshold 612. It should be appreciated that, in some instances, the first patient activity threshold 608 and the second patient activity threshold 612 may be the same value. After the patient’s activity has been above the first and/or second patient activity threshold 608, 612 for the activity time period threshold, at time T_{A}, a frequency analysis flag 614 may be triggered, switching the medical device 100 into the frequency mode 506. A frequency mode tracking signal 616 may track the time elapsed between the activity time period threshold being exceeded and the end of the maximum time-out period 520 (e.g., the time period between T_{A} and T_{Max}).

The medical device 100 is configured to continue to monitor the patient activity signal 606 throughout the frequency mode 506. As illustrated, if the patient activity signal 606 drops below the first and/or second patient activity threshold 608, 612, the frequency analysis flag 614 may switch off momentarily, temporarily suspending the frequency analysis of the patient’s movements. However, as long as the patient activity signal 606 rises back above the first and/or second patient activity threshold 608, 612 within a grace period, the frequency analysis flag 614 may switch back on, resuming the frequency analysis of the patient’s movements. Alternatively, if the patient activity signal 606 remains below the first and/or second patient activity threshold 608, 612 for longer than the grace period, as long as the patient remains in the recumbent position, the patient activity timer 610 may end and the patient inactivity timer 604 may begin again.

In some instances, the grace period may be pre-programmed into the medical device 100. In other instances, the grace period may be selected by a healthcare professional via the external computing system 24, the external monitoring device 122, and/or the remote device 126.

As shown in FIG. 6, the medical device 100 detects a seizure event, at time T_{Detect}, and the frequency analysis flag 614 switches off, ending the frequency analysis of the patient’s movements. The patient activity timer 610 then ends, at time T_{Max}, and the refractory period 524 begins. The refractory period 524 may last until a refractory timer 618 has concluded.

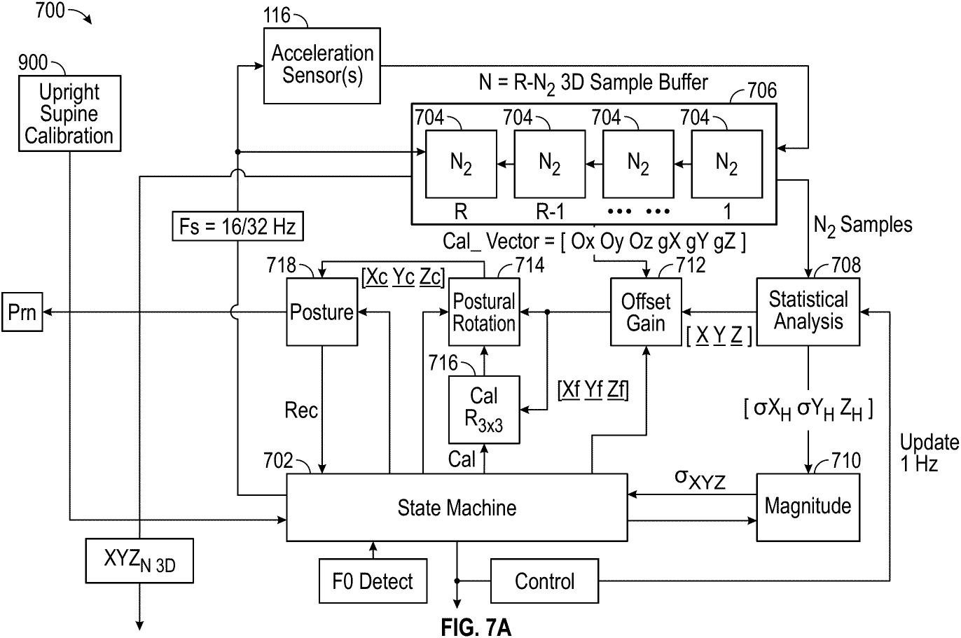

Referring now to FIGS. 7A-7C, an exemplary flow diagram 700 is shown depicting the process for the medical device 100 obtaining and analyzing the acceleration data obtained from the acceleration sensor 116, according to an exemplary embodiment.

As shown in FIG. 7A, a first portion of the flow diagram 700 depicts various data analysis blocks utilized in controlling the acceleration sensor 116 and analyzing the patient’s posture and activity level, according to an exemplary embodiment. For example, the medical device 100 includes a state machine 702 (e.g., stored on the memory 106 or the seizure detection circuit 108 of the controller 102) configured to control operation of the acceleration sensor 116 and each of the various analysis blocks of the flow diagram 700. The acceleration sensor 116 may obtain N number of samples 704, at a sample acquisition block 706, at a sampling frequency of either 16 Hz (e.g., when determining the patient’s posture and/or activity level) or 32 Hz (e.g., when performing the frequency analysis of the patient’s movement data). For example, in some instances, the frequency analysis may require the higher sample rate of 32 Hz to allow for the correct assessment of abnormal activity in the tonic-clonic or clonic band of frequencies while assuring the event is most likely not a tonic seizure or normal activity.

The state machine 702 may then determine the mean, RMS, and standard deviation values within a given sampling window, at a statistical analysis block 708. In some instances, the determined mean, RMS, and standard deviation values may be recursively determined (e.g., utilizing a modified moving average) based, in part, on values obtained from one or more previous sampling windows. In some instances, the sampling window may be four seconds long (i.e., containing data from the previous four seconds) and each sampling window may be obtained one second after the previous sampling window, such that there is a 75% overlap between adjacent sampling windows. In other instances, the sampling window may be longer or shorter than four seconds and each sampling window may be obtained at a different time interval, such that there is a larger or smaller overlap between adjacent sampling windows, as desired for a given application.

Further, in some instances, the state machine 702 is configured to obtain data from an X channel, a Y channel, and a Z channel of the acceleration sensor 116 recursively in sequence (i.e., the X channel during a first sampling window, the Y channel during a subsequent sampling window, the Z channel during a subsequent sampling window, etc.). In some other instances, the state machine 702 may be configured to obtain data from each of the X, Y, and Z channels simultaneously for each sampling window.

The method for estimating standard deviation from each channel of the acceleration data may be an approximation to the exact calculation of standard deviation or RMS after removal of the mean. It is very efficient to generate this result by low pass filtering the absolute value of the data over the appropriate integration time. The method becomes exact when the noise data is assumed to be Gaussian since the absolute value of a Gaussian process becomes a Half-Normal process. The averaging operation due to the low pass filter that follows effectively approximates the standard deviation to within a well-known multiplicative constant with a Half-Normal distribution. The reciprocal of this constant may then be used to rescale and correct the result of the magnitude calculation.

For example, the X, Y, and Z channel RMS values and/or standard deviations (σX_{H}, σY_{H}, σZ_{H}) are output from the statistical analysis block 708 to a magnitude approximation block 710 by the state machine 702. The magnitude approximation block 710 is configured to determine an absolute magnitude of the RMS value and/or standard deviation (σ_{xyz}) using the X, Y, and Z channel RMS values and/or standard deviations (σX_{H}, σY_{H}, σZ_{H}). This absolute magnitude of the RMS value and/or standard deviation (σ_{xyz}) is then provided to the state machine 702 to be used in activity monitoring and/or frequency analysis.

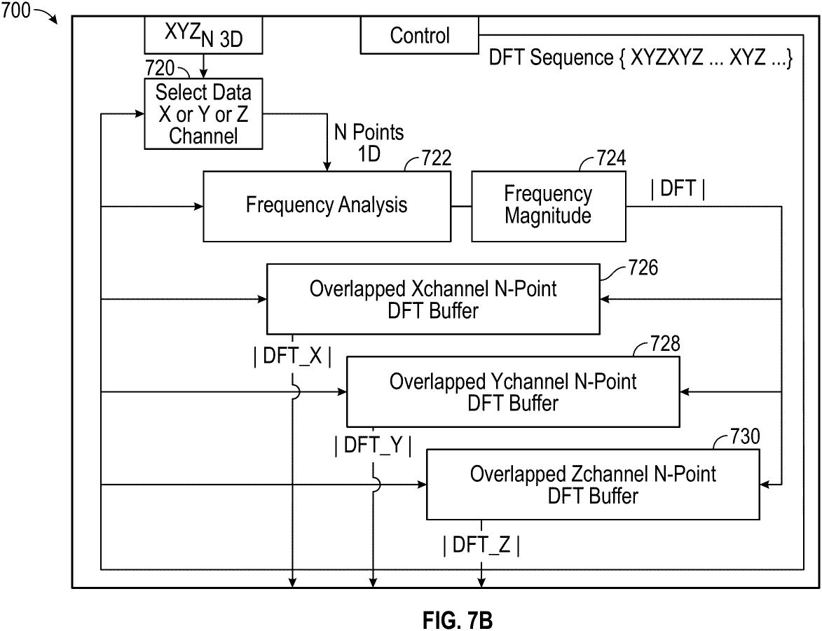

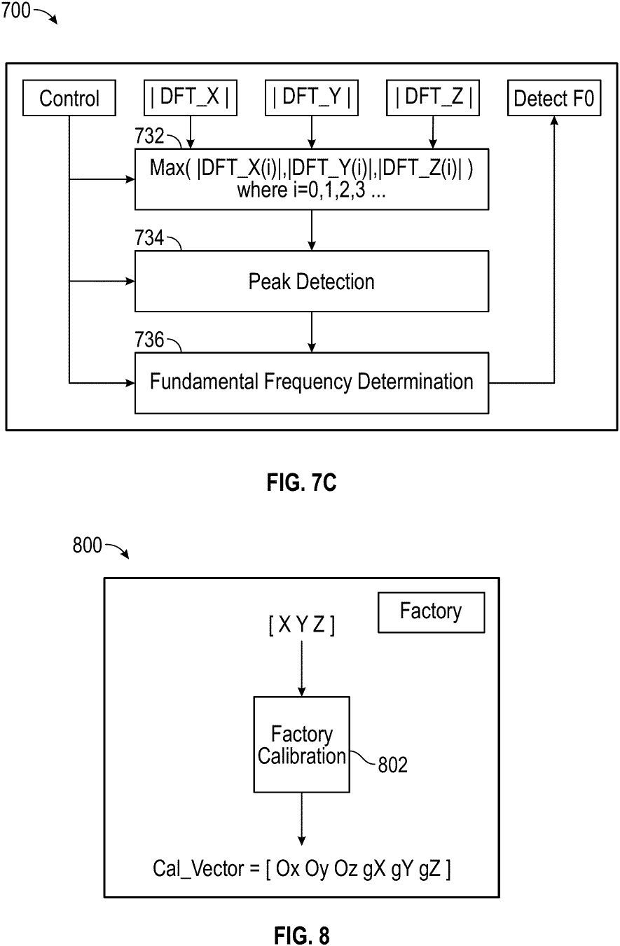

The determined mean values for the X, Y, and Z channels (X, Y, Z) are then output to an offset and gain correction block 712 to apply an offset and gain correction and then a postural rotation block 714 to orient the data for patient posture determination. For example, in order to accurately assess the patient’s posture the acceleration sensor 116 must go through two calibration procedures: a factory calibration procedure 800 (shown in FIG. 8) and an upright and supine calibration procedure 900 (shown in FIG. 9).

As shown in FIG. 8, the factory calibration procedure 800 is a six point calibration where gain and offset values are extracted for each axis of the acceleration sensor 116 in a manufacturing environment. For example, during the factory calibration procedure 800, the acceleration sensor 116 is placed in a factory test fixture such that X, Y, and Z channels (i.e., the three orthogonal acceleration directions) of the acceleration sensor 116 are exposed to ± 1 G fields and the offset and gains are calculated for each channel during a factory calibration block 802. Accordingly, the factory calibration block 802 generates a calibration vector including an offset and a gain for each of the X, Y, and Z channels (Ox, O_{Y}, Oz, gX, gY, gZ). The 6 values of the calibration vector are then stored in the medical device 100 for use during operation.

Accordingly, as shown in FIG. 7A, the state machine 702 is configured to perform an offset and gain correction, at block 714, to correct the acceleration data received from the acceleration sensors 116 for the gain and offset errors within the acceleration sensor 116. For example, the offset and gain correction block 712 may receive the determined mean values for the X, Y, and Z channels (X, Y, Z) and apply the calibration vector to them, at block 902, to obtain factory-corrected mean values for the X, Y, and Z channels (Xf, Yf, Zf).

It should be appreciated that, during the offset and gain correction, at block 712, only one subtraction of the offset, one multiplication by the multiplicative inverse, and one associated arithmetic shift is performed for each data collection window for the X, Y, and Z channels, thereby further reducing the processing requirements of the medical device 100. This also avoids any need for a divide in the medical device 100 (e.g., a separate flow path of data) at this step.

Referring now to FIG. 9, prior to performing the method 400, the state machine 702 is configured to perform the upright and supine calibration procedure 900 to obtain a rotation calibration matrix Cal R configured to correct any rotation alignment errors due to the implant position of the medical device 100 (or rotation alignment errors due to a typical worn location/orientation of the medical device 100 if it is to be worn externally on the patient’s body), which may then be used to determine a postural rotation matrix R.

Specifically, during the upright and supine calibration procedure 900, acceleration data is captured by the acceleration sensor 116 while the patient is in an upright position, at block 902, and a supine position, at block 904. The state machine 702 is then configured to determine the mean values of the X, Y, and Z data obtained by the acceleration sensors 116 for the upright data, at block 906, and the supine data, at block 908. The state machine 702 is then configured to apply the stored calibration vector from the factory calibration procedure 800 to the mean values for the upright position, at block 910, and the supine position, at block 912, to correct for the offset and gain errors. The corrected mean values are then combined to create the rotation calibration matrix Cal R, at block 914. This rotation calibration matrix Cal R may then be stored by the state machine 702 (e.g., in the memory 106 or the seizure detection circuit 108 of the medical device 100) to be used to create the postural rotation matrix R during operation.

Accordingly, referring back to FIG. 7A, the factory-corrected mean values for the X, Y, and Z channels (Xf, Yf, Lf) may be applied to the rotation calibration matrix Cal R, provided by the state machine 702, at a rotational calibration matrix block 716, to create the postural rotation matrix R. The postural rotation matrix R may then be applied to the factory-corrected mean values for the X, Y, and Z channels (Xf, Yf, Zf) at the postural rotation block 714 to obtain the final corrected mean values for the X, Y, and Z channels (Xc, Yc, Zc) to be used to determine the patient’s posture at a posture determination block 718.

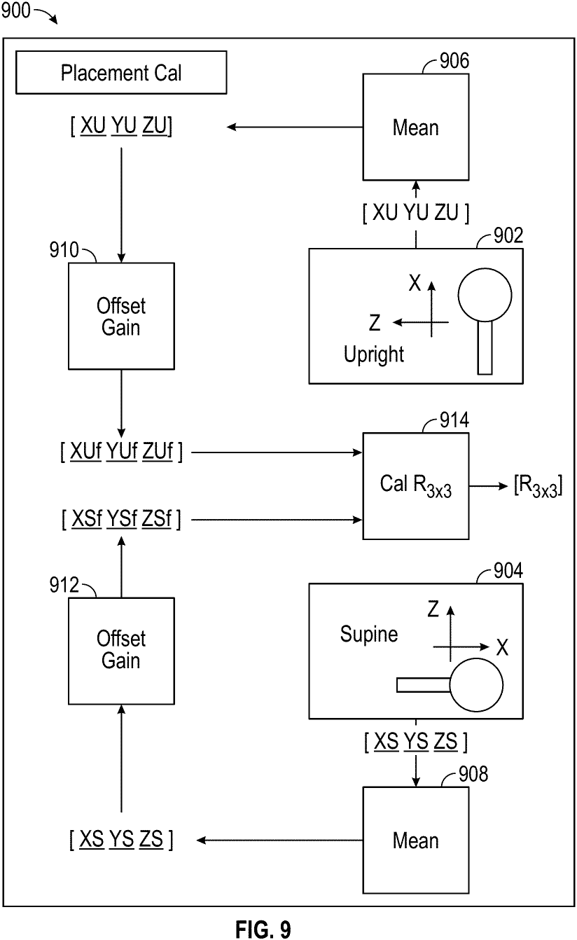

At the posture determination block 718, the state machine 702 is configured to perform a posture detection procedure 1000, as shown in FIG. 10, to determine whether the patient is in a recumbent position and/or a prone position. Specifically, referring to FIG. 10, the state machine 702 is configured to determine, based on the final corrected mean values for the X, Y, and Z channels (Xc, Yc, Zc), whether the patient is in a recumbent position, at step 1002. If the patient is not in a recumbent position, the state machine 702 may also determine that the patient is also not in a prone position. Alternatively, if the patient is in a recumbent position, the state machine 702 is then configured to determine, based on the rotated and corrected mean data vector, whether the patient is in a prone position, at step 1004. In any case, the determined patient posture (Rec Prn) is provided back to the state machine 702 to be utilized in controlling the medical device 100 (e.g., performing the multi-stage MEOI detection algorithm), as described herein.

As such, the state machine 702 may utilize the postural information determined in FIG. 7A to determine when to monitor the patient’s activity level and perform the frequency analysis, as described above, with respect to FIGS. 4-6.

It should be appreciated that the various blocks associated with the postural determination (e.g., blocks 712, 714, 716, 718) described above may be omitted while assessing the patient’s activity levels and/or performing the frequency analysis. The omission of these operations during the assessment of the patient’s activity levels and/or the frequency analysis further reduces the processing requirements of the medical device 100.

Referring now to FIGS. 7B and 7C, second and third portions of the flow diagram 700 depict various analysis blocks utilized in performing the frequency analysis, according to an exemplary embodiment. For example, once the state machine 702 has triggered the frequency analysis, as described above with respect to FIGS. 4-6, the sampled X, Y, and Z channel data may be provided to a data selection block 720. At the data selection block 720, the state machine 702 may select sampled data from the X, Y, and/or Z data channels for the frequency analysis.

In some instances, as discussed above, the frequency analysis may be performed using the X channel data for a first sampling window, the Y channel data for a second sampling window, the Z channel data for a third sampling window, and so on. In these instances, due in part to the 75% overlap between sampling windows, this sequential analysis allows for an accurate assessment of the most pertinent spectral contributions, while reducing the overall computational requirements imposed on the medical device 100 (e.g., to one third of the computational requirements involved in performing the frequency analysis using the X, Y, and Z channel data simultaneously), thus reducing the required power consumption of the medical device 100. However, it should be appreciated that, in some other instances, the frequency analysis may be performed using the X, Y, and Z channel data simultaneously.

For each sampling window, a frequency analysis is performed on the sampled data, at a frequency analysis block 722. In some instances, the frequency analysis may be a traditional discrete Fourier transform (DFT) and/or a fast Fourier transform (FFT) analysis. In other instances, the frequency analysis may be a specialized radix-2 real decimation in time (DIT) FFT analysis employing recursively processed DFTs employing specialized 2-point, 3-point, and 4-point butterfly operations configured to allow for a significant reduction in complex rotations required as compared to traditional DFT and FFT analyses.

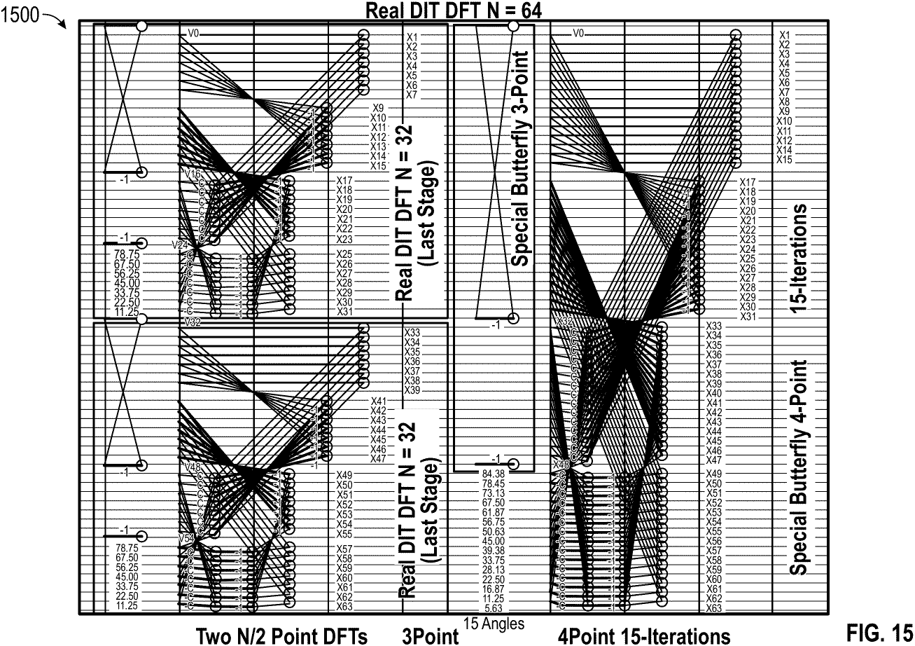

For example, referring to FIGS. 11-16, in some instances, various calculations utilizing the specialized radix-2 real DIT FFT may be performed in place to minimize storage requirements. Further, the inputs and outputs of the specialized radix-2 real DIT FFT are both real. In some instances, the specialized radix-2 real DIT FFT is a 128-point real DIT FFT (i.e., a real DIT FFT where N=128), as shown in FIG. 16. In these instances, a 32 entry cosine table lookup may be used (a separate sine table is not needed). The following discussion will provide explanations of 4-, 8-, 16-, 32-, 64-, and 128-point radix-2 real DIT FFTs. However, it should be appreciated that the recursive frequency analysis techniques described herein is expandable to any set of data where N (the number of samples in a given sample window) = 2^{M}, where M is an integer with a value of 2 or more.

In any case, the radix-2 real DIT FFT is configured to generate an N-point output vector comprising real values [X(0), X(1) ... X(N/2)] in ascending order followed by imaginary values [X(N/2-1) ... X(2), X(1)] in descending order. It should be appreciated that both X(0) and X(N/2) in this output vector are real values. It should further be appreciated that, although the depicted radix-2 real DIT FFT is shown with respect to the X channel, the same real DIT FFT is configured to be similarly applied to the Y and/or Z channels, as well as corresponding data sets generated using three-dimensional absolute magnitude vectors obtained from the X, Y, and Z channel data.

Referring to FIG. 11, an exemplary specialized 4-point radix-2 real DIT discrete Fourier transform (DFT) flow graph 1100 is shown, according to an exemplary embodiment. For real data, traditional 4-point DFTs produce an output sequence such that X(0) and X(2) are real, while X(1) and X(3) are complex. However, for real data, the information contained in X(1) and X(3) are redundant since the spectrum is conjugate odd symmetric. In place calculation is thus afforded by saving X(0) and X(2) in place while saving the real content of X(1) in place and the imaginary content of X(1) in the usual location for X(3) without any loss of information. For example, in some instances, these values (e.g., X(0), X(1), X(2), X(3)) are all saved as real-valued items regardless of whether their content comes from either the real components or the imaginary components of items that otherwise could have been saved as complex.

As will be described below, there are 3 key steps utilized for the 4-point radix-2 real DIT DFT. Repetitions of the flow graph 1100 are used repeatedly for longer N-point radix-2 real DIT DFT analyses where N = 8, 16, 32, 64, and 128 with different sets of 0-based bit reversed inputs.

For example, as illustrated in FIG. 11, in a first step, a four-data-point input vector [R0, R1, R2, R3] is reordered according to the 0-based bit-reversed index method, thereby generating a first intermediate vector [X0, X2, X1, X3]. It should be appreciated that, while the C language software utilized in this example utilizes 0-based array indexing, various other programing software utilize 1-based array indexing. Accordingly, the same techniques described herein may be similarly applied to 1-based array indexing programs.

During a second step, the even index points of the first intermediate vector (e.g., X0 and X2) are sent to a top special 2-point butterfly operation, while the odd index points of the first intermediate vector (e.g., X1 and X3) are sent to a bottom special 2-point butterfly operation. These operations output a second intermediate vector [U0, U1, U2, U3]. It should be appreciated that the special 2-point butterfly operations used in the second step only require simple sum and difference operations (i.e., no multiplications are required). Subtracted quantities are indicated by -1 at the bottom lines of the top and bottom 2-point butterfly operations.

During a third step, a special 3-point butterfly operation is utilized to output a final output vector [V0, U1, V2, V3]. The special 3-point butterfly operation utilized in the special 4-point radix-2 real DIT DFT analysis (as well as the expanded special radix-2 real DIT DFT analyses discussed below) applies only simple sum and difference operations at V(0) and V(N/2) (e.g., V0 and V2 in FIG. 11) and only a simple sign change for V(3N/4) (e.g., V3 shown in FIG. 11).

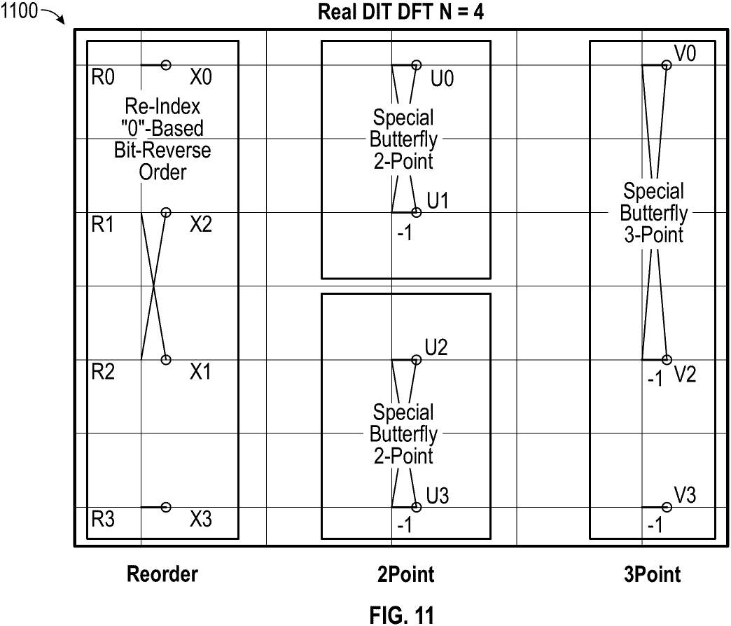

Referring now to FIG. 12, an exemplary 8-point real DIT DFT flow graph 1200 is shown, according to an exemplary embodiment. The 8-point real DIT DFT draws on two N/2-point (e.g., 4-point) real DIT DFTs from the previously-described recursion operation (i.e., shown in FIG. 11). In a traditional 8-point radix-2 DFT, the final stage uses four 2-point butterfly operations, each involving complex rotations (twiddle factors). However, a careful examination of the traditional radix-2 DFT shows that the last butterfly operation is redundant for real data. Accordingly, in the specialized 8-point radix-2 real DIT DFT flow graph 1200, the real part of the 8-point DFT outputs X(0) and X(4) (e.g., V0 and V4 in FIG. 12) are saved in place and the real parts of X for indices [1, 2, 3] (e.g., X1, V2, X3 in FIG. 12) are saved in place while the imaginary components X for indices [5, 6, 7] (e.g., X5, V6, X7) are saved in reverse order respectively. This allows processing to progress in place and without redundant temporary storage.

In the 8-point real DIT DFT flow graph 1200, an even bit-reverse indexed input vector [X0, X4, X2, X8] produces a first N/2 (4-point) DFT output vector [V0, U1, V2, V3], as described above, with respect to the 4-point real DIT DFT flow graph 1100. An odd bit-reverse indexed input vector [X1, X5, X3, X7] produces a second N/2 (4-point) DFT output vector [V4, U5, V6, V7], as described above, with respect to the 4-point real DIT DFT flow graph 1100.

These two N/2 point DFT output vectors [V0, U1, V2, V3] and [V4, U5, V6, V7] are input to a final stage which consists of another application of the special 3-point butterfly described above and the first usage of a special 4-point butterfly.

As depicted in FIG. 12, the 8-point radix-2 real DIT DFT final output values X1, X3, X5, X7 are determined as a function of U1, V3, U5 (rotated), and V7 (rotated). Specifically, the 4-point butterfly operation is driven using a single rotation where ⊖=45 degrees. That is, the nodes U5 and V7 are passed through a 45-degree rotation step generating temporary values T1 and T2 (rotated versions of U5 and V7). As shown in FIG. 12, in this recursion for N=8, C=cos(⊖) and S=sin(⊖), where ⊖= 45 degrees. The nodes T1 and U1 are used to update X1 and X3, while the nodes T2 and V3 are used to update X5 and X7.

It should be appreciated that, in the 8-point real DIT DFT flow graph 1200, only 1 complex rotation is required. The traditional butterfly flow graph of an 8-point DFT for complex data utilizes 4 complex rotations (twiddle factors) in the final stage. This reduction in complex rotations affords a significant savings in multiplications and additions, thereby reducing the overall processing requirements of the medical device 100, and thus the power consumption requirements of the medical device 100. As will be described below, this reduction in the overall processing requirements and power consumption requirements of the medical device 100 is more apparent when analyzing data sets with higher sample sizes (e.g., N = 16, 32, 64, 128, etc.).

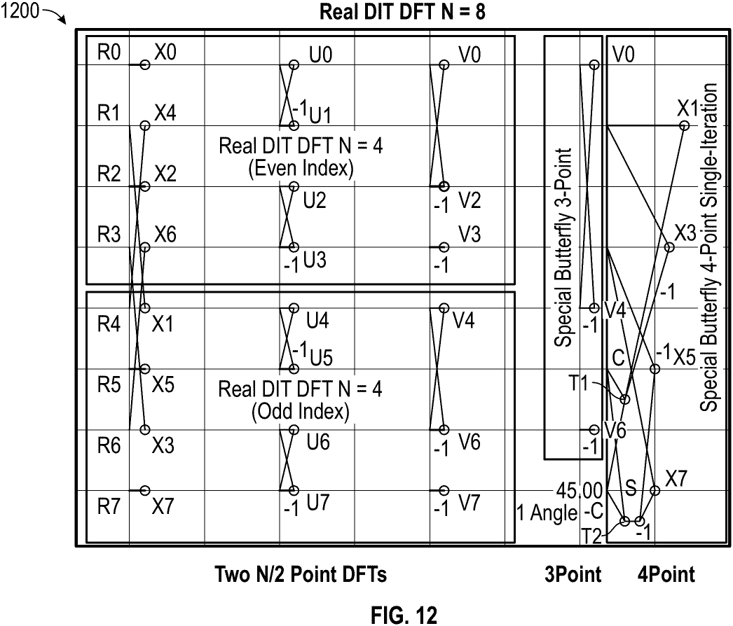

Referring now to FIG. 13, an exemplary 16-point radix-2 real DIT DFT flow graph 1300 is shown, according to an exemplary embodiment. This 16-point radix-2 real DIT DFT draws on two N/2 point (8-point) radix-2 DFTs from the previous recursion (e.g., shown in FIG. 12).

Comparing the topology of the 16-point radix-2 real DIT DFT flow graph 1300 to the previous 8-point real DIT DFT flow graph 1200, there are two repetitions of the 8-point real DIT DFT, but with a different set of even and odd indexed inputs. The outputs of the two 8-point radix-2 real DIT DFTs drive the 16-point radix-2 real DIT DFT calculation. In order to focus on the indexing associated with iterating the rotation angles applied in the 16-point real DIT DFT calculation, only the last 2 stages (i.e., the final 3-point and 4-point butterfly operations) of the 16-point real DIT DFT flow graph 1300 are discussed below.

Specifically, first, the special 3-point butterfly operation is performed as described above, with respect to the 3-point butterfly operation of the 4-point radix-2 real DIT DFT, such that the nodes V0, V8, and V12 (i.e., V(0), V(N/2), and V(3N/4)) are updated by the 3-point butterfly operation.

After the 3-point butterfly operation, the 4-point butterfly operation is repeated 3 times where ⊖= 22.5 degrees, 45 degrees, and 67.5 degrees. That is, 3 sets of connection lists may be handled iteratively by rotating appropriate nodes and using the rotated nods to update various output values. Specifically, X9 and X15 are first rotated by ⊖= 22.5 degrees, producing temporary variables T1 and T2. Once temporary variables T1 and T2 are produced, T2 (rotated version of X15) and X7 are used to update output values X9 and X15, and T1 (rotated version of X9) and X1 are used to update output values X1 and X7.

Next, nodes V10 and V14 are rotated by ⊖= 45 degrees, producing new temporary variables T3 and T4. Once new temporary variables T3 and T4 are produced, T4 (rotated version of V14) and V6 are used to update output values X10 and X14, and T3 (rotated version of V10) and V2 are used to update output values X2 and X6.

Finally, nodes X11 and X13 are rotated by ⊖= 67.5 degrees, producing new temporary variables T5 and T6. Once new temporary variables T5 and T6 are produced, T6 (rotated version of X13) and X5 are used to update output values X11 and X13, and T5 (rotated version of X11) and X3 are used to update output values X3 and X5. Accordingly, after the third rotation angles are applied and used to update the final output values, the 16-point radix-2 DIT DFT can output a final output vector [V0, X1, X2, X3, V4, X5, X6, X7, V8, X9, X10, X11, V12, X13, X14, X15], as shown in FIG. 13.

It should be appreciated that the 16-point real DIT DFT calculation described above only requires 3 complex rotations. The traditional flow graph for the final stage of a 16-point DTF utilizes 8 complex rotations in the final stage. This reduction in complex rotations (i.e., eliminating 5 complex rotations) again affords a significant savings in multiplications, thereby further reducing the overall processing requirements of the medical device 100, and thus the power consumption requirements of the medical device 100.

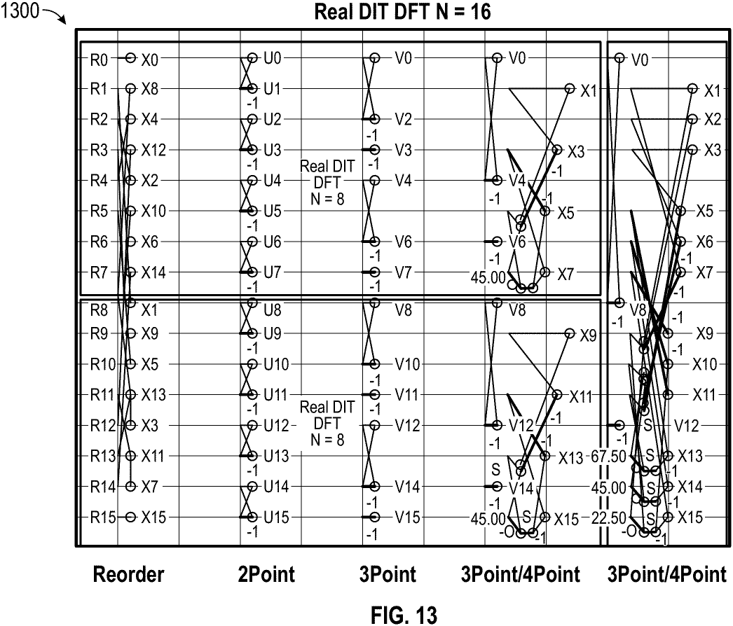

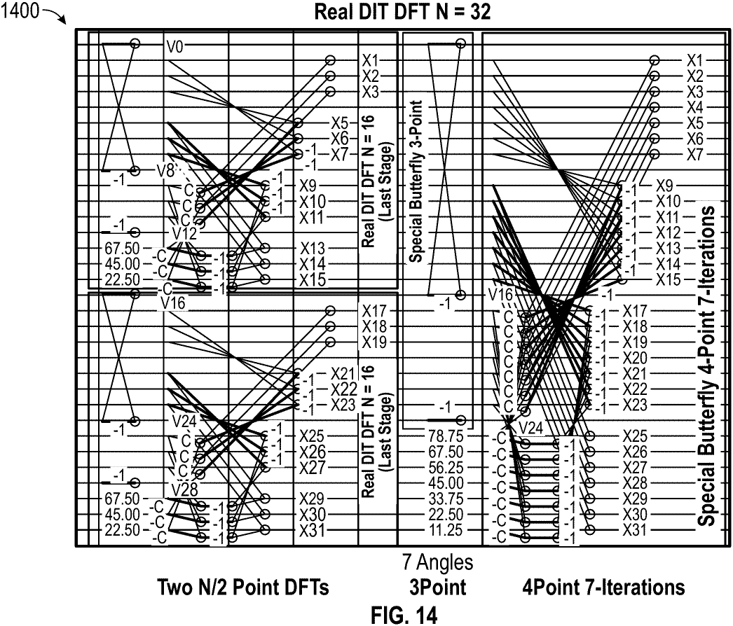

As discussed above, it should be appreciated that the special radix-2 real DIT DFT calculations described above may be expanded to any set of data, where N = 2^{M} and M = 1, 2, 3, 4, etc., to allow for a reduction in complex rotation calculation requirements. For example, as shown in FIG. 14, a 32-point real DIT DFT flow graph 1400 shows that a 32-point radix-2 real DIT DTF calculation may utilize two 16-point real DIT DFT calculations, as discussed above, and combine them into a single output vector using a similar three-point butterfly operation and similar four-point iterative butterfly operations, as discussed above, requiring only 7 total complex rotations applied iteratively. It should be appreciated that the traditional flow graph for the final stage of a 32-point DTF utilizes 16 complex rotations in the final stage. Similarly, as shown in FIG. 15, a 64-point radix-2 real DIT DFT flow graph 1500 shows that a 64-point real radix-2 DIT DTF calculation may utilize two 32-point radix-2 real DIT DFT calculations and combine them into a single output vector using a similar three-point butterfly operation and similar four-point iterative butterfly operations, as discussed above, requiring only 15 total complex rotations applied iteratively. It should be appreciated that the traditional flow graph for the final stage of a 64-point DTF utilizes 32 complex rotations in the final stage.

In some instances, the medical device 100 is configured to utilize a 128-point radix-2 real DIT FFT analysis of the X, Y, and/or Z channel data at the frequency analysis block 722, as shown by the 128-point real DIT FFT flow graph 1600 in FIG. 16, to obtain an output frequency vector. The 128-point radix-2 real DIT FFT analysis may utilize two 64-point real DIT DFT calculations and combine them into a single output vector using a similar three-point butterfly operation and similar four-point iterative butterfly operations, as described above, requiring only 31 total complex rotations applied iteratively. It should be appreciated that the traditional flow graph for the final stage of a 128-point DTF utilizes 64 complex rotations in the final stage. As such, this drastic reduction in complex rotations (i.e., 33 fewer complex rotations) significantly reduces the multiplication steps necessary for the specialized recursive 128-point radix-2 real DIT FFT analysis described herein as compared to a traditional 128-point DTF analysis. As such, the specialized recursive 128-point real DIT FFT described herein beneficially allows for a reduction in processing requirements of the medical device 100, and thus the power consumption requirements of the medical device 100.

Referring again to FIG. 7B, after the output frequency vector has been obtained for the X, Y, or Z data, an X, Y, or Z frequency magnitude |DFT_X|, |DFT_Y|, |DFT_Z| of the corresponding output frequency vector is determined, at a frequency magnitude determination block 724. The determined X, Y, or Z frequency magnitude |DFT_X|, |DFT_Y|, |DFT_Z| for each sampling window of a detection period is then sent to and compiled within a corresponding overlapped X channel buffer block 726, overlapped Y channel buffer block 728, or overlapped Z channel buffer block 730.

Referring now to FIG. 7C, the state machine 702 is configured to determine the maximum X, Y, or Z frequency magnitude at each frequency, bin-by-bin, from the most recent valid X, Y, Z spectra, at a maximum magnitude, bin-by-bin, at each frequency determination block 732. This single vector of maximum magnitudes is then assessed for the 3 most significant frequency bins (i.e., having the highest amplitude) above a preset amplitude threshold and within a preset frequency band of interest, at a peak detection block 734. For example, in some instances, the preset amplitude threshold may be 1.3 times the median spectral amplitude estimate. In some instances, the preset frequency band of interest may extend from 1.5 Hz to 10 Hz. This preset band of interest may be selected because, in addition to the frequency range of 2-5 Hz in the clonic seizure phase, various amounts of higher-frequency energy in the range of 5-10 Hz have been observed in some instances in the tonic interval preceding the clonic interval of a tonic-clonic seizure.

The highest 3 resulting amplitudes and associated frequencies are retained as long all 3 amplitude values are above 0.25 times the maximum amplitude value of the 3 amplitude values. Accordingly, in some instances, there may be 1, 2 or 3 surviving frequency bins. The fundamental frequency of the patient’s movement is then determined, at a fundamental frequency determination block 736. For example, the fundamental frequency determination block 736 may determine the fundamental frequency by identifying the lowest frequency of the up to 3 surviving frequencies as the fundamental frequency.

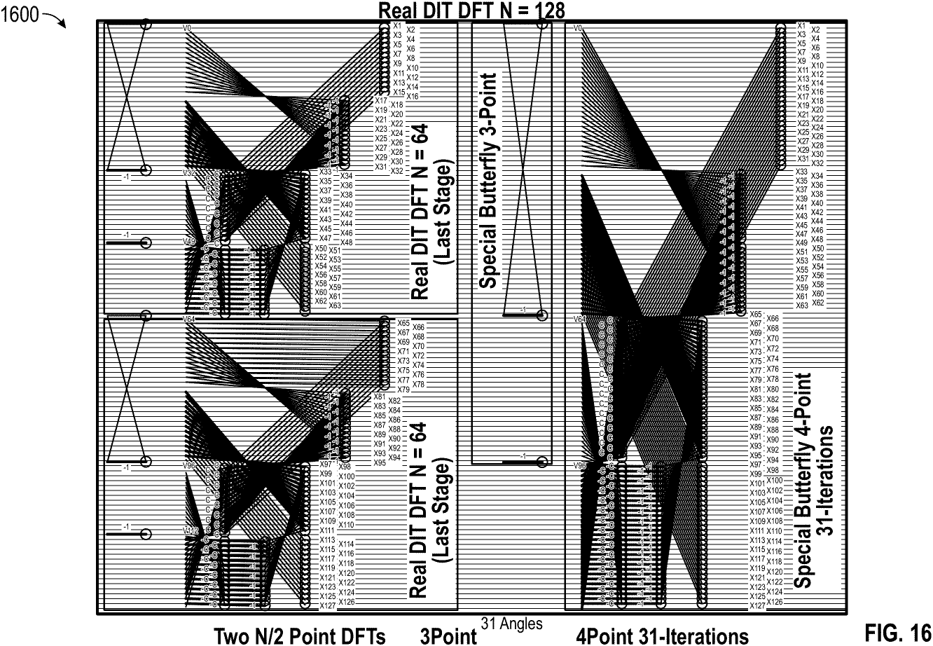

Referring now to FIG. 17, a frequency plot 1700 created based on the fundamental frequency assessment performed at blocks 732, 734, and 736 is shown, according to an exemplary embodiment. As illustrated, a frequency line 1702 tracks the maximum X, Y, or Z frequency magnitudes at each frequency within the preset frequency band of interest (e.g., a frequency range 1704). The fundamental frequency F₀ is identified as the lowest frequency peak of the top 3 amplitude peaks (P₁, P₂, P₃) above a threshold line 1706 that is set as the higher value of 1.3 times a median spectral estimate 1708 or 0.25 times the maximum peak amplitude, as discussed above. In some instances, there may be only be 1 or 2 amplitude peaks above the threshold line 1706. In these instances, the fundamental frequency F₀ is identified as the lowest frequency peak of the 1 or 2 amplitude peaks above the threshold line 1706. In the exemplary embodiment shown in FIG. 17, the identified fundamental frequency F₀ is within a clonic range 1710 of movement frequencies. As such, the state machine 702 may determine that the patient is experiencing a clonic, tonic-clonic, or generalized tonic-clonic seizure event.

It should be noted that it is possible that the data may reveal that the extracted fundamental frequency lies in a band above the maximum or below the minimum acceptable frequency range. It is also possible that the fundamental frequency may not appear as the frequency with the highest amplitude. Both considerations are within the scope of the present disclosure.

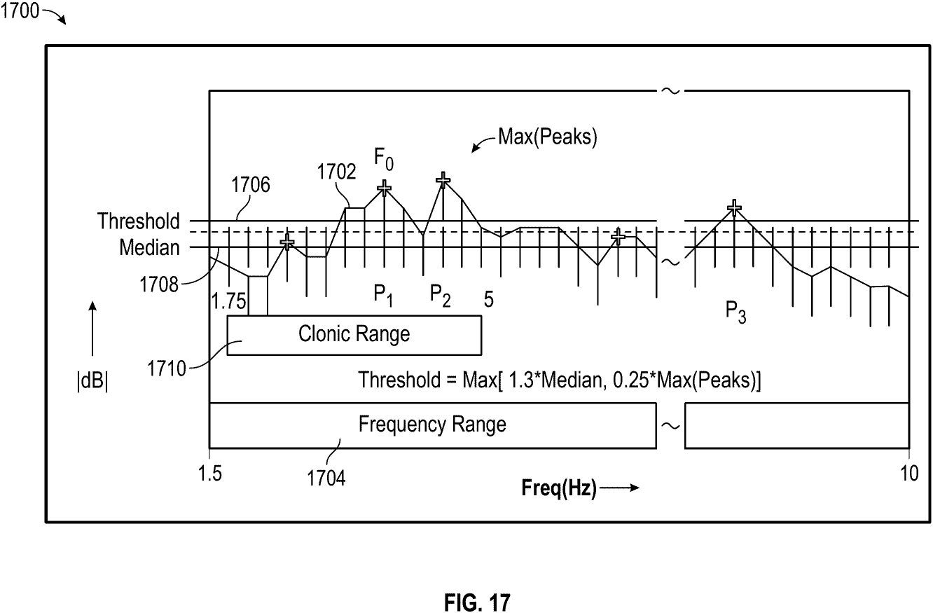

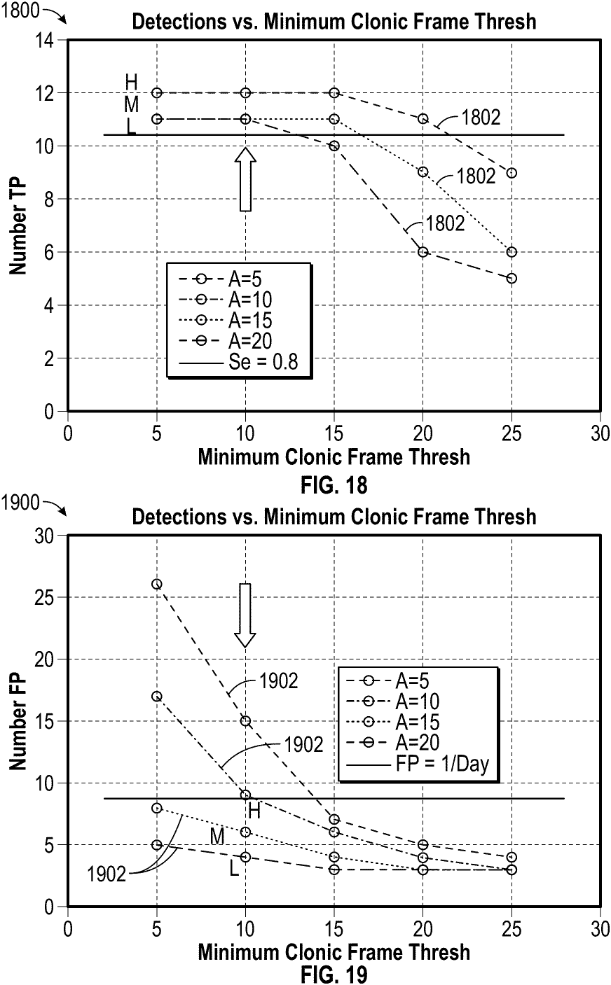

Referring now to FIGS. 18 and 19, a plot 1800 depicting the number of true positive seizure detections 1802 (FIG. 18) and a plot 1900 depicting the number of false positive seizure detections 1902 (FIG. 19) across a range of differing minimum clonic frame thresholds (e.g., differing minimum seizure detection periods) for various differing activity time period thresholds are shown, according to an exemplary embodiment. For example, as discussed above, in some instances, the multi-stage MEOI algorithm may be configured such that a healthcare professional may select the activity time period threshold and/or the minimum seizure detection period via the external computing system 24, the external monitoring device 122, and/or the remote device 126 to support both fine tuning for optimal seizure detection performance and patient/doctor individual preferences.

As illustrated in FIGS. 18 and 19, there is a trade-off between raising and/or lowering the activity time period threshold and/or the minimum seizure detection period and the resulting success of detecting true positives and avoiding false positives. For example, as depicted in FIG. 18, as either the activity time period threshold or the minimum seizure detection period are reduced, the number of successful detections of true positive seizure events is increased. Conversely, as depicted in FIG. 19, as either the activity time period threshold or the minimum seizure detection period are reduced, the number of detections of false positive seizure events is also increased.

It should be appreciated that, although the multi-stage MEOI detection algorithm described herein is discussed in reference to various medical devices 12, 13, 100 for detecting seizure events, it should be appreciated that the multi-stage MEOI detection algorithm may be provided as a standalone algorithm for use with various medical other systems, devices, servers, etc., and for analysis of a variety of fundamental patient movements of interest. For example, in some instances, remote recording of streams of sensor data can be processed using the multi-stage MEOI detection algorithm in real-time or offline (i.e., non-real-time).

The present disclosure contemplates methods, systems, and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general purpose computer, special purpose computer, or special purpose processing machines to perform a certain function or group of functions.

Although the figures may show a specific order of method steps, the order of the steps may differ from what is depicted. Also two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, Z, X and Y, X and Z, Y and Z, or X, Y, and Z. Thus, such conjunctive language is not generally intended to imply that certain embodiments require at least one of X, at least one of Y, and at least one of Z to each be present.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed:

1. A seizure detection system comprising:
a control system programmed to:
analyze collected acceleration data associated with a patient’s movements to detect a seizure precursor event;
upon detection of the seizure precursor event, perform a frequency analysis of the acceleration data to identify a frequency of the patient’s movements; and
determine that the patient is experiencing a seizure event based on an identification that the frequency of the patient’s movements indicates the seizure event.

2. The seizure detection system of claim 1, wherein detecting the seizure precursor event comprises:
determining that the patient has been in a recumbent position for longer than a recumbent time period threshold;
upon determining that the patient has been in the recumbent position for longer than the recumbent time period threshold, determining that the patient has been inactive for longer than an inactivity time period threshold; and
upon determining that the patient has been inactive for longer than the inactivity time period threshold, determining that the patient has been active for longer than an activity time period threshold.

3. The seizure detection system of claim 2, wherein the control system is further programmed to:
receive feedback from the patient pertaining to an experienced seizure event; and
adjust at least one of the recumbent time period threshold, the inactivity time period threshold, or the activity time period threshold based on the feedback.

4. The seizure detection system of claim 1, wherein detecting the seizure precursor event comprises detecting a patient fall event.

5. The seizure detection system of claim 1, wherein the control system is an implantable medical device.

6. The seizure detection system of claim 5, wherein the implantable medical device is a chest-based medical device.

7. The seizure detection system of claim 1, wherein the control system is further programmed to:
upon determination that the patient is experiencing the seizure event, provide a notification to an external computing device, the notification indicating that the patient is experiencing the seizure event.

8. The seizure detection system of claim 1, wherein the frequency analysis is a real decimation in time (DIT) fast Fourier transform (FFT) analysis employing at least one special 3-point butterfly operation and at least one special 4-point butterfly operation collectively configured to reduce a number of complex rotations involved in the real DIT FFT analysis.

9. The seizure detection system of claim 8, wherein the real DIT FFT analysis is a 128-point real DIT FFT analysis involving only 31 total complex rotations.

10. The seizure detection system of claim 1, further comprising a stimulation circuit configured to deliver therapeutic stimulation to a nerve of the patient, and wherein the control system is further programmed to:
upon determining that the patient is experiencing the seizure event, deliver, via the stimulation circuit, the therapeutic stimulation to the nerve of the patient.

11. A method of detecting a seizure event of a patient, the method comprising:
analyzing collected acceleration data associated with the patient’s movements to detect a seizure precursor event;
upon detection of the seizure precursor event, performing a frequency analysis of the acceleration data to identify a frequency of the patient’s movements; and
determining that the patient is experiencing the seizure event based on an identification that the frequency of the patient’s movements indicates the seizure event.

12. The method of claim 11, wherein detecting the seizure precursor event comprises:
determining that the patient has been in a recumbent position for longer than a recumbent time period threshold;
upon determining that the patient has been in the recumbent position for longer than the recumbent time period threshold, determining that the patient has been inactive for longer than an inactivity time period threshold; and
upon determining that the patient has been inactive for longer than the inactivity time period threshold, determining that the patient has been active for longer than an activity time period threshold.

13. The method of claim 12, further comprising:
receiving feedback from the patient pertaining to an experienced seizure event; and
adjusting at least one of the recumbent time period threshold, the inactivity time period threshold, or the activity time period threshold based on the feedback.

14. The method of claim 11, wherein detecting the seizure precursor event comprises detecting a patient fall event.

15. The method of claim 11, wherein the sensor is located within one of an implantable medical device implanted in a chest of the patient or a wearable medical device worn on the chest of the patient.

16. The method of claim 11, wherein the frequency analysis is a real decimation in time (DIT) fast Fourier transform (FFT) analysis employing at least one special 3-point butterfly operation and at least one special 4-point butterfly operation collectively configured to reduce a number of complex rotations involved in the real DIT FFT analysis.

17. The method of claim 16, further comprising:
upon determining that the patient is experiencing the seizure event, delivering, via a stimulation circuit, therapeutic stimulation to a nerve of the patient.

18. One or more non-transitory computer-readable mediums comprising instructions executable by one or more processors to:
analyze collected acceleration data associated with a patient’s movements to detect a seizure precursor event;
upon detection of the seizure precursor event, perform a frequency analysis of the acceleration data to identify a frequency of the patient’s movements;
determine that the patient is experiencing a seizure event based on an identification that the frequency of the patient’s movements indicates the seizure event.

19. The one or more non-transitory computer-readable mediums of claim 18, wherein detecting the seizure precursor event comprises:
determining that the patient has been in a recumbent position for longer than a recumbent time period threshold;
upon determining that the patient has been in the recumbent position for longer than the recumbent time period threshold, determining that the patient has been inactive for longer than an inactivity time period threshold; and
upon determining that the patient has been inactive for longer than the inactivity time period threshold, determining that the patient has been active for longer than an activity time period threshold.

20. The one or more non-transitory computer-readable mediums of claim 18, wherein the instructions are further executable by the one or more processors to:
upon determining that the patient is experiencing the seizure event, deliver, via a stimulation circuit, therapeutic stimulation to a nerve of the patient.
